(19) United States
(12) Patent Application Publication
(10) Pub. No.: US 2007/0203079 A1
(43) Pub. Date: Aug. 30, 2007

(54) Methods of using small molecule compounds for neuroprotection

(76) Inventors: Guy A. Caldwell, Northport, AL (US); Kim A. Caldwell, Northport, AL (US); Songsong Cao, Northport, AL (US)

Correspondence Address: KING & SPALDING LLP, 1180 PEACHTREE STREET, ATLANTA, GA, 30309-3521, US

(21) Appl. No.: 11/603,924

(22) Filed: Nov. 21, 2006

Related U.S. Application Data
(60) Provisional application No. 60/738,761, filed on Nov. 21, 2005.
(60) Provisional application No. 60/749,910, filed on Dec. 12, 2005.

(51) Int. Cl. A61K 31/7048; A61K 31/704; A61K 31/4741; A61K 31/4745; A61K 31/404

(52) U.S. Cl. 514/27; 514/34; 514/414; 514/283; 514/292; 514/569

(57) ABSTRACT

Methods are provided for preventing neurodegeneration and neuronal loss by administering compositions comprising small molecule compounds with the effect of preventing neurodegeneration and neuronal loss. In one aspect of the invention, the methods and compositions are also useful for treating neurodegenerative diseases. Small molecule compounds provide an important treatment option because of their stability, ease of use in both manufacture and formulation, ease of administration, and patient compliance. The small molecule compound compositions of the present invention may include topoisomerase II inhibitors, bacterial transpeptidase inhibitors, calcium channel antagonists, cyclooxygenase inhibitors, folic acid synthesis inhibitors, or sodium channel blockers and functional analogues thereof that have an effect on neurodegeneration. The compositions of the present invention may be administered prophylactically before the onset of clinical symptoms or after clinical symptoms of a neurodegenerative disease have manifested.

### CROSS-REFERENCE TO RELATED APPLICATIONS

The present application claims the benefit of U.S. Provisional Patent Application Ser. No. 60/738,761, filed Nov. 21, 2005, and U.S. Provisional Patent Application Ser. No. 60/749,910, filed Dec. 12, 2005, both of which are incorporated herein by reference in their entirety.

### FIELD OF THE INVENTION

The present invention relates to methods and compositions comprising small molecule compounds for protecting neurons from death or degeneration due to central nervous system injury or disease.

### BACKGROUND OF THE INVENTION

Disease and injury of the central nervous system (“CNS”) cause devastating debilitating conditions that alter the lives of millions of individuals each year. Generally, these conditions develop after neuron death and degeneration that results in mild to severe clinical manifestation of a disease or disorder. Injury from trauma, ischemia, and many other insults of neuropathological origin are known to cause neuronal damage and death either directly or indirectly through mechanisms such as oxidative stress, free radical damage, or malfunction of cellular proteins. Examples of CNS injuries or disease include traumatic brain injury (“TBI”); posttraumatic epilepsy (“PTE”); stroke; cerebral ischemia; neurodegenerative diseases; brain injuries secondary to seizures, induced by radiation, exposure to ionizing or iron plasma, nerve agents, cyanide, or toxic concentrations of oxygen; neurotoxicity due to CNS malaria or treatment with anti-malaria agents; and other CNS traumas. Both CNS neuronal injury and neurodegenerative disease often result in further neuronal loss due to apoptosis, oxidative stress, and mitochondrial dysfunction.

Neurodegenerative diseases are characterized by progressive loss of neurons and are associated with (1) enzyme dysfunction, (2) the formation of reactive oxygen species, and/or (3) protein misfolding and aggregation that ultimately lead to tissue degeneration. Neurodegenerative diseases include, among others, Parkinson's disease, Alzheimer's disease, Huntington's disease, amyotrophic lateral sclerosis (“ALS”), polyglutamine diseases, tauopathy, dystonia, spinocerebellar ataxia, spinal and bulbar muscular atrophy, and spongiform encephalopathies—including prion diseases.

Neuronal injury and disease may result from enzyme dysfunction. Many cellular enzymes are critical to the function of neurons and alterations in protein function can be devastating to cell survival. Normal metabolic enzymes recycle proteins creating a perpetual cycle of synthesis and degradation. Cellular enzymes responsible for normal cell function include receptors, neurotransmitter transporters, synthesis and degradation enzymes, molecular chaperones and transcription factors. Mutations in these enzymes result in abnormal accumulation and degradation of misfolded proteins. These misfolded proteins are known to result in neuronal damage such as neuronal inclusions and plaques. Therefore, the understanding of the cellular mechanisms and the identification of the molecular tools for the reduction, inhibition, and amelioration of such misfolded proteins is critical. Furthermore, an understanding of the effects of protein aggregation on neuronal survival will allow the development of rational and effective treatment protocols for these disorders.

Formation of neurotoxic reactive oxygen species appear to both initiate pathways for cellular/neuronal degeneration and play a significant role in mediating necrotic neuronal death. Specific toxins may be used in vivo to screen for compounds that protect neurons from reactive oxygen species damage and neurodegeneration. For example, toxins that cause formation of excessive reactive oxygen species and induce dopaminergic neuron loss and Parkinsonian phenotypes in animal models include 1-methyl-4-phenyl-1,2,3,6-tetrahydropyridine (“MPTP”), paraquat, rotenone, and 6-hydroxydopamine (“6-OHDA”) (Simon et al., Exp Brain Res, 1974, 20: 375-384; Langston et al., Science, 1983, 219: 979-980; Tanner, Occup Med, 1992, 7: 503-513; Liou et al., Neurology, 1997, 48: 1583-1588).

Onset of ALS is commonly spontaneous and the roles of trace metals and reactive oxygen species are also implicated in sporadic cases of ALS and other neurodegenerative diseases such as Alzheimer's disease, Parkinson's disease, prion diseases, polyglutamine diseases, spinocerebellar ataxia, spinal & bulbar muscular atrophy, spongiform encephalopathies, tauopathy, and Huntington's disease (Manfredi and Xu, Mitochondrion, 2005 April, 5(2): 77-87; Zeevalk et al., Antioxid Redox Signal, 2005 September-October, 7(9-10): 1117-1139).

TorsinA is a protein that belongs to the functionally diverse AAA+ protein superfamily of ATPases that includes heat shock proteins (“Hsp”), proteases, and dynein (Neuwald et al., Genome Res., 1999, 9: 27-43). The torsin family of proteins possessing molecular chaperone activity includes torsinA, torsinB, TOR-1, TOR-2, and OOC-5. TorsinA was recently shown to modulate cellular levels of the dopamine transporter (“DAT”) and other polytopic membrane-bound proteins (Tones et al., Proc Natl Acad Sci USA, 2004, 101: 15650-15655). TorsinA is believed to be neuroprotective to dopaminergic neurons after exposure to reactive oxygen species by modulation of the DAT (Cao et al., J Neurosci, 2005, 25(1): 3801-3812). Reduction or loss of torsin protein activity also abrogates its capacity to modulate protein folding and may result in protein aggregation and neurodegeneration in response to adverse environmental conditions. Mutations in the torsinA protein have also been directly linked to early-onset torsion dystonia, a human movement disorder (L. J. Ozelius, et al., Nature Genetics, 1997, 17: 40).

Molecular chaperone proteins, such as torsin proteins, are among the normal cellular proteins that prevent protein misfolding and aggregation. Molecular chaperone proteins include protein families such as Hsp100, Hsp90, Hsp70, Hsp60, and Hsp40 (Muchowski and Wacker, Nature Reviews, 2005, 6: 11-22). Mutations in human torsinA result in early-onset torsion dystonia, a movement disorder characterized by uncontrolled muscle spasms. The symptoms can range in severity from a writer's cramp to being wheelchair bound. Dystonia affects more than 300,000 people in North America and is more common than Huntington's disease and muscular dystrophy. Treatment is very limited because the disease is poorly understood and options include surgery or injection of botulism toxin to control the muscle contractions.

The majority of patients with early onset torsion dystonia have a unique deletion of one codon of the torsinA gene (“DYT1”), which results in a loss of glutamic acid (“GAG”) residue at the carboxy terminal of torsinA thereby producing a dysfunctional torsin protein (L. J. Ozelius, et al., Genomics, 1999, 62: 377; L. J. Ozelius, et al., Nature Genetics, 1997, 17: 40). A recent paper described an additional deletion of 18 base pairs or 6 amino acids at the carboxy terminus that may also result in early onset torsion dystonia (Leung, et al., Neurogenetics, 2001, 3: 133-143).

Torsin proteins have also been implicated in preventing protein misfolding and aggregation in diseases of polyglutamine expansion and α-synuclein misfolding related to neurodegenerative diseases such as Huntington's disease and Parkinson's disease (Caldwell et al., Hum Mol Genetics, 2003, 12: 307-319; Cao et al., J Neurosci, 2005, 25: 3801-3812) (See also Cooper et al., Science, 2006, 313: 324-328). Neurodegenerative disorders such as Parkinson's disease, Huntington's disease, and polyglutamine expansion diseases result from abberant protein misfolding and aggregation. Torsin proteins have been shown to ameliorate protein misfolding and aggregation in in vivo models of these disorders. It is believed that torsin proteins also have actions on other proteins implicated in neurodegenerative diseases associated with protein misfolding and aggregation.

A major obstacle surrounding neurodegenerative disorders is that patients are unaware that a neuronal environment contributing to neuronal degeneration is developing until the point where clinical symptoms manifest. By the time clinical symptoms become apparent, there is already substantial neuronal loss and the neuronal environment is significantly hostile to the survival of neurons. Genetic screening provides information on whether or not an individual is predisposed to developing a neurodegenerative disease. However, the lack of reliable early detection methods for protein aggregation or neuronal loss allows these degenerative diseases to develop unmonitored until a point where treatment may be ineffective or unnecessary as neuronal loss has already occurred. Furthermore, even if reliable early detection methods were available, current therapies are ineffective for long-term treatment of these neurodegenerative diseases and novel drugs and treatment methods are necessary.

A better understanding of molecular mechanisms and regulators of aberrant protein aggregation is necessary in order to develop improved methods for early stage diagnosis of resulting disorders prior to significant neuronal destruction, and for guiding drug design and development. Compounds that target specific genes and gene products related to protein aggregation may be screened for, and developed, using model systems. In addition, it is also necessary to understand the mechanisms of neurodegeneration and develop neuroprotective compounds that may prevent or attenuate protein misfolding and aggregation and ensuing neuronal loss.

The prevalence of CNS injury and disease highlights the need for an improved understanding of the mechanisms of development and progression of neurodegeneration. It is also apparent that a need exists for novel and improved neuroprotective compounds for preventing or attenuating neuronal loss either prophylactically or after injury and disease manifestation. What is therefore needed are novel therapeutics for protecting neurons from death and degeneration due to CNS injury or disease.

What is also needed are novel therapeutics for treating and preventing diseases resulting from neuronal damage, including protein misfolding and protein aggregation. Ideally, such therapeutics would have prophylactic use as well as utility following onset of symptoms. Currently available therapeutic options include vaccines and protein therapies that are both difficult to produce and to administer. While such therapeutics may provide treatment options where none exist, the difficulty in manufacturing and administration may result in low patient compliance. Therefore, what is also needed are therapeutics that are easy to produce and administer and result in high patient compliance.

### SUMMARY OF THE INVENTION

Methods are provided for protecting neurons from damage and death due to injury, ischemia, or neurodegeneration by administering small molecule compounds with the effect of preventing neuronal death. In one aspect of the present invention, these methods are useful for treating neuronal damage and neurodegenerative diseases associated with dysfunctional cellular proteins. In another aspect of the present invention, these methods are also useful for treating neuronal damage and neurodegenerative diseases associated with reactive oxygen species. In a further aspect of the present invention, these methods are useful for preventing and reducing protein misfolding or aggregation in vitro or in vivo by administering small molecule compounds. Another aspect of the present invention provides methods for treating neuronal damage and neurodegenerative diseases associated with protein misfolding and aggregation.

The small molecule compounds of the present invention include topoisomerase II inhibitors, bacterial transpeptidase inhibitors, calcium channel antagonists, cyclooxygenase inhibitors, folic acid synthesis inhibitors, or sodium channel blockers and functional analogues thereof that have a neuroprotective effect. The neuroprotective effect may be a result of modulating cellular proteins such as neurotransmitter transporters or molecular chaperone proteins. The small molecule compounds may act by modulating torsin protein activity that reduces neuronal damage due to defective cellular proteins. The small molecule compounds may also act by modulating torsin protein activity that reduces neuronal damage due to reactive oxygen species by regulating neurotransmitter transporter molecules on the surface of neurons. The small molecule compounds may further act to modulate torsin protein molecular chaperone activity that reduces neuronal damage due to protein misfolding or aggregation by helping to guide the proper folding of proteins. Small molecule compounds provide an important treatment option because of their stability, ease of use in both manufacture and formulation, ease of administration, and patient compliance. The compounds may be administered prophylactically before the onset of clinical symptoms or after clinical symptoms of a CNS injury or neurodegenerative disease have manifested.

Accordingly, it is an object of the present invention to provide methods and compositions for protecting neurons from injury or death after CNS injury or neurodegeneration.

It is another object of the present invention to identify small molecule compounds for use in methods and compositions for treating or preventing neurodegeneration and neuronal loss associated with defective cellular proteins.

It is another object of the present invention to identify small molecule compounds for use in methods and compositions for treating or preventing neurodegeneration and neuronal loss associated with reactive oxygen species.

It is a further object of the present invention to identify small molecule compounds for use in methods and compositions for treating or preventing neurodegeneration and neuronal loss associated with protein misfolding and aggregation.

It is another object of the present invention to provide methods and compositions for preventing neurodegeneration and neuronal loss by using small molecule compounds that include topoisomerase II inhibitors, bacterial transpeptidase inhibitors, calcium channel antagonists, cyclooxygenase inhibitors, folic acid synthesis inhibitors, or sodium channel blockers and functional analogues thereof.

It is another object of the present invention to identify small molecule compounds for use in methods and compositions for treating or preventing neurodegeneration and neuronal loss by modulating the actions of torsin proteins.

It is a further object of the present invention to provide methods for treating or preventing neurodegeneration and neuronal loss associated with protein misfolding and aggregation by modulating the activity of torsin proteins.

It is another object of the present invention to identify small molecule compounds that act through torsin-dependent mechanisms for use in methods and compositions for treating or preventing neurodegeneration and neuronal loss associated with protein misfolding and aggregation.

It is another object of the present invention to identify small molecule compounds that modulate neurotransmitter transporter molecule activity for use in methods and compositions for treating or preventing neurodegeneration and neuronal loss associated with protein misfolding and aggregation.

These and other objects, features, and advantages of the present invention will become apparent after a review of the following detailed description of the disclosed embodiments and the appended claims.

### BRIEF DESCRIPTION OF THE DRAWINGS

FIG. 1 provides a graph showing the effects of candidate drugs identified in a primary screen of the Prestwick library. FIG. 1a shows 7 candidate drugs that reduce the incidence of protein misfolding and aggregation in a C. elegans model of neurodegenerative disease due to polyglutamine aggregation. FIG. 1b shows that two of the 7 compounds work directly on the aggregated protein through torsin-independent mechanisms. FIG. 1c shows that 3 compounds work through torsin-dependent mechanisms. FIG. 1d shows that 2 drugs work by acting on dysfunctional torsinA. Compounds are identified by their number in the Prestwick library.

FIG. 2 provides a graph showing the effects of functionally-related compounds within each class of drugs identified in the primary screen of the Prestwick library. Compounds are identified by their number in the Prestwick library.

FIG. 3a provides a graph showing the effects of a preventative assay for the 5 candidate compounds acting through torsin-dependent mechanisms when the drug is administered from hatching to L2 stage. The hatched bars represent the standardized decrease in protein aggregation at L3 larval stage and the solid bars represent the standardized decrease in protein aggregation at the young adult stage. Compounds are identified by their number in the Prestwick library. FIG. 3b shows the preventative assay format including drug exposure and withdrawal and time of assay for aggregate reduction.

FIG. 4a provides a graph showing the effects of a corrective assay for the 5 candidate compounds acting through torsin-dependent mechanisms when the drug exposure is from the L2 stage onward. The hatched bars represent the standardized decrease in protein aggregation at L3 larval stage and the solid bars represent the standardized decrease in protein aggregation at the young adult stage. Compounds are identified by their number in the Prestwick library. FIG. 4b shows the corrective assay format including drug exposure and withdrawal and time of assay for aggregate reduction.

FIG. 5a provides a graph showing the effects of 3 candidate compounds on preventing dopaminergic neuron damage due to 6-OHDA insult. The compounds are identified by their number in the Prestwick library (50-lidocaine HCl; 206-meclofenamic acid sodium salt monohydrate; 23 5-metampicillin sodium salt). FIG. 5b provides a graph showing the effects of the same compounds from FIG. 5a in a torsin-independent model and shows that 2 of the 3 compounds protect dopaminergic neurons from damage through a torsin-independent mechanism. FIGS. 5c and 5d provide graphs showing that the actions of metampicillin sodium salt (Prestwick compound 235) on preventing dopaminergic neurodegeneration are through a torsin-dependent mechanism. Metampicillin provides neuroprotection in transgenic worms expressing wild type (“wt”) torsinA protein. Transgenic worms expressing a mutant torsinA (ΔE) are not protected by metampicillin after 6-OHDA insult.

FIG. 6 provides a graph showing the effects of mafenide (Prestwick compound 66) and meclofenamic acid sodium salt monohydrate (Prestwick compound 206) on preventing the occurrence of neurodegeneration due to overproduction of dopamine.

FIG. 7 provides a graph showing the effects of a group of compounds related to metampicillin sodium salt (Prestwick compound 235) on preventing neurodegeneration due to 6-OHDA insult in a C. elegans model of neurodegeneration.

FIG. 8 provides a graph showing that wild-type (“wt”) torsinA can prevent dopamine neuron degeneration resulting from overexpression of α-synuclein in the dopaminergic neurons of C. elegans, while mutant torsinA has a reduced neuroprotection.

FIG. 9 provides a graph showing the torsinA-specificity of 5 torsinA-dependent compounds from the Prestwick library that reduce the incidence of protein misfolding and aggregation in a C. elegans model of neurodegenerative disease. FIG. 9 shows that 3 of the 5 Prestwick compounds act specifically on wild-type (“wt”) torsinA protein to enhance dopaminergic neuron survival through torsinA-dependent mechanisms. FIG. 9 further shows that 2 of the 5 Prestwick compounds act specifically on mutant torsinA protein to enhance dopaminergic neuron survival through torsinA-dependent mechanisms.

### DETAILED DESCRIPTION OF THE INVENTION

It is understood that methods and materials similar or equivalent to those described herein can be used in the practice or testing of the present invention. All publications, patent applications, patents, and other references mentioned herein are incorporated by reference in their entirety. In case of conflict, the present specification, including definitions, will control. In addition, the materials, methods, and examples are illustrative only and are not intended to be limiting.

“Small molecule compounds,” “candidate compounds,” and “drug compounds” refer to the molecular compounds of the present invention screened for an effect on formation of reactive oxygen species, protein misfolding and aggregation, neuronal injury, and neurodegeneration. These compounds may comprise compounds in the Prestwick library, in related drug classes, or functional analogues thereof.

“Protein misfolding” refers to folding of a protein that is different than the normal manner that a protein folds to achieve a secondary or tertiary structure. Errors in protein folding may result from changes in the protein sequence due to mutation or from defects in molecular chaperone proteins that aid in proper protein folding. Misfolding may cause altered physiological function of a protein that may increase, decrease, or prevent proper protein function.

“Protein aggregation,” within the scope of the present invention, refers to the abnormal association of polypeptides to form assemblies of self-associated states, which may be soluble or insoluble, and not necessarily fibrillary. This term also includes the phenomenon of at least two polypeptides contacting each other in a manner that causes either one of the polypeptides to be in a state of de-solvation. This may also include a loss of the polypeptide's native functional activity.

“Treating,” within the scope of the present invention, refers to reducing, inhibiting, ameliorating, or preventing. Preferably, neurodegeneration due to reactive oxygen species, cellular dysfunction as a result of reactive oxygen species, neurodegenerative diseases, protein misfolding, protein aggregation, cellular dysfunction as a result of protein misfolding and aggregation, and protein-aggregation-associated diseases may be treated.

“Protein-aggregation-associated disease,” within the scope of the present invention, includes any disease, disorder, and/or affliction associated with, caused by, or resulting in protein-aggregation-associated disease—including neurodegenerative disorders.

“Neurodegenerative disorders” comprise disorders resulting from neuronal loss due to etiological factors that result in progressive degradation of sensory, motor, and cognitive behavior. Such disorders comprise ALS; Alzheimer's disease; Parkinson's disease; prion diseases; polyglutamine expansion diseases; spinocerebellar ataxia; spinal and bulbar muscular atrophy; spongiform encephalopathies; tauopathy; Huntington's disease; frontotemporal dementia; motor neuron disease (“MND”); and the like.

“CNS injuries” include traumatic brain injury (“TBI”); posttraumatic epilepsy (“PTE”); stroke; cerebral ischemia; neurodegenerative diseases; brain injuries secondary to seizures, induced by radiation, exposure to ionizing or iron plasma, nerve agents, cyanide, or toxic concentrations of oxygen; neurotoxicity due to CNS malaria or treatment with anti-malaria agents; and the like.

As used herein, “analogue” or “functional analogue” refers to a chemical compound that is structurally similar to a parent compound, but differs slightly in composition (e.g., one atom or functional group is different, added, or removed). The analogue may or may not have different chemical or physical properties than the original compound and may or may not have improved biological and/or chemical activity. For example, the analogue may be more hydrophilic or it may have altered reactivity as compared to the parent compound. The analogue may mimic the chemical and/or biologically activity of the parent compound (i.e., it may have similar or identical activity), or, in some cases, may have increased or decreased activity. The analogue may be a naturally or non-naturally occurring (e.g., recombinant) variant of the original compound. Other types of analogues include isomers (enantiomers, diasteromers, and the like) and other types of chiral variants of a compound, as well as structural isomers. The analogue may be a branched or cyclic variant of a linear compound. For example, a linear compound may have an analogue that is branched or otherwise substituted to impart certain desirable properties (e.g., improve hydrophilicity or bioavailability).

As used herein, “derivative” refers to a chemically or biologically modified version of a chemical compound that is structurally similar to a parent compound and (actually or theoretically) derivable from that parent compound. A “derivative” differs from an “analogue” or “functional analogue” in that a parent compound may be the starting material to generate a “derivative,” whereas the parent compound may not necessarily be used as the starting material to generate an “analogue” or “functional analogue.” A derivative may or may not have different chemical or physical properties of the parent compound. For example, the derivative may be more hydrophilic or it may have altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” also includes conjugates, and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al., Advanced Drug Delivery Reviews 19 (1996) 115; Design of Prodrugs, H. Bundgaard (ed.), Elsevier, 1985; and H. Bundgaard, Drugs of the Future 16 (1991) 443. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups such as carboxylic acid groups can form alkali metal salts or alkaline earth metal salts (e.g., sodium salts, potassium salts, magnesium salts, calcium salts, and salts with physiologically tolerable quaternary ammonium ions and acid addition salts with ammonia and physiologically tolerable organic amines such as triethylamine, ethanolamine, or tris-(2-hydroxyethyl)amine). Basic groups can form acid addition salts, for example with inorganic acids such as hydrochloric acid (“HCl”), sulfuric acid, or phosphoric acid, or with organic carboxylic acids and sulfonic acids such as acetic acid, citric acid, benzoic acid, maleic acid, fumaric acid, tartaric acid, methanesulfonic acid, or p-toluenesulfonic acid. Compounds which simultaneously contain a basic group and an acidic group such as a carboxyl group in addition to basic nitrogen atoms can be present as zwitterions. Salts can be obtained by customary methods known to those skilled in the art, for example by combining a compound with an inorganic or organic acid or base in a solvent or diluent, or from other salts by cation exchange or anion exchange.

Protein-aggregation-associated diseases all share a conspicuous common feature: aggregation and deposition of abnormal protein (Table 1), and the role of molecular chaperone proteins has also been implicated in such diseases (Muchowski and Wacker, Nature Reviews, 2005, 6:11-22). Protein-aggregation-associated diseases include, but are not limited to, Alzheimer's disease, Parkinson's disease, polyglutamine disease, tauopathy, Huntington's disease, dystonia, spnocerebellar ataxia, spinal and bulbar muscular atrophy, spongiform encephalopathies, and ALS. Expression of mutant proteins in transgenic animal models recapitulates features of these diseases (A. Aguzzi and A. J. Raeber, Brain Pathol, 8, 695 (1998)). Neurons are particularly vulnerable to the toxic effects of mutant or misfolded protein. As described herein, an understanding of the common characteristics related to protein-aggregation-associated neurodegenerative disorders, such as an understanding of the normal cellular mechanisms for disposing of unwanted and potentially noxious proteins and promoting the proper folding of proteins, enables the development of efficient and successful therapeutic regimens and diagnostic methods.

TABLE 1
Protein aggregation-associated neurodegenerative diseases

| | Protein | | | |
|---|---|---|---|---|
| Disease | deposits | Toxic Protein | Diseases gene | Risk factor |
| Alzheimer's | Extracellular | Amyloid 13 | APP | ApoE4 allele |
| disease | plaques | | Presenilin 1 | |
| | Intracellular | tau | Presenilin 2 | |
| | tangles | | | |
| Parkinson's | Lewy bodies | Alpha- | Alpha- | Tau linkage |
| disease | | synuclein | synuclein | |
| | | | Parkin | |
| | | | UCHL-1 | |
| | | | LRRK2 | |
| Prion disease | Prion plaque | PrP^{Sc} | PRNP | Homozygosity |
| | | | | at pnon codon |
| | | | | 129 |
| Polyglutamine | Nuclear and | Polyglutamine- | 9 different | |
| disease | cytoplasmic | containing | genes with | |
| | inclusions | proteins | CAG repeat | |
| | | | expansion | |
| Tauopathy | Cytoplasmic | tau | tau | tau linkage |
| Familial | Tangles Bunina | SOD1 | SOD1 | |
| amyotrophic | bodies | | | |
| lateral sclerosis | | | | |

Correct folding requires proteins to assume one particular structure from a constellation of possible but incorrect conformations. The failure of polypeptides to adopt their proper structure is a major threat to cell function and viability. Consequently, elaborate systems have evolved to protect cells from the deleterious effects of misfolded proteins. The first line of defense against misfolded proteins includes the molecular chaperones, which associate with nascent polypeptides as they emerge from the ribosome, promoting correct folding and preventing harmful interactions (J. P. Taylor, et al., Science, 2002, 296: 1991). Improper folding of proteins may not necessarily result in protein aggregation or neurodegeneration, but clinical symptoms of a disease or disorder may still manifest due to more subtle causes of cellular dysfunction. For example, in early-onset torsion dystonia, a defective torsinA protein does not properly modulate cellular levels of the dopamine transporter thereby resulting in dystonic symptoms without apparent neurodegeneration or protein aggregation (Torres et al., Proc Natl Acad. Sci., 2002, 101: 15650-15655; Cao et al., J Neurosci, 2005, 25(1): 3801-3812).

The present inventors screened a chemically diverse small molecule library to identify small molecule compounds with actions on preventing protein misfolding and aggregation (Table 2). The C. elegans small molecule library was obtained from Prestwick Chemical, Inc. (Washington, D.C.) (hereafter “Prestwick Library”). The library is a chemically diverse collection of 240 small molecules that have been selected for tolerability in C. elegans. All compounds in the library have been examined for toxicity over the lifetime of the worms and have been shown to be non-toxic for C. elegans. The candidate compounds do not color or obscure the incubation medium for histological studies. Over 95% of the compounds in the library are off-patent marketed drugs and have been safely administered to humans. Approximately 5% of these drugs are alkaloids. This library permits screening for drugs that provide hits with sufficient potency. If a positive result occurs but without sufficient potency, optimized analogs may be synthesized using computer-assisted drug design and readily screened in the same manner. The compounds in this library provide opportunities for further development since the initial lead is non-toxic, orally bioavailable with an acceptable half-life, and is well tolerated.

To facilitate the chances of success, compounds may be selected using computer programs such as ChemX/ChemDiverse (Accelrys) and based on critical devices by experienced medicinal chemists. To increase the diversity and increase the success of the screening process, compounds with known efficacy in different therapeutic areas may be assembled in this library. These compounds may include neuropsychiatry, anti-diabetic, antiviral, antihypertensive, antipyretic, anti-inflammatory, antibiotic, and anti-infective drugs.

Screening the small molecule library in a C. elegans model for protein aggregation has identified several classes of compounds with an effect on preventing protein misfolding and aggregation (Table 2). Drugs may be plated onto substrates where the transgenic worms are grown or administered in other conventional manners to expose the worms to the candidate drugs. When introduced into the growth substrate, the small molecule compounds penetrate the animals both by diffusion through the cuticle and ingestion. This mode of administration allows the continuous exposure of animals to the drug. When drugs produce a response at the initial screening concentration, serial dilutions were made to define the highest possible dose to cause the observed effect.

Expression of mutant proteins in transgenic animal models recapitulates features of neurodegenerative diseases (A. Aguzzi and A. J. Raeber, Brain Pathol, 1998, 8: 695). Neurons are particularly vulnerable to the toxic effects of mutant or misfolded proteins. The common characteristics of these neurodegenerative disorders suggest parallel approaches to treatment, based on an understanding of the normal cellular mechanisms for preventing damage due to reactive oxygen species.

C. elegans is an ideal model for studying the degeneration of dopaminergic neurons because this anatomically and genetically defined transparent nematode has exactly 302 neurons, 8 of which are dopaminergic (“DA”). Accordingly, use of the C. elegans model facilitates rapid scoring of dopamine neurodegeneration while the animal ages. Dopaminergic neurons are particularly susceptible to oxidative stress as a result of dopamine metabolism, as well as the presence of other intracellular factors favoring the formation of reactive oxygen species (Blum et al., 2001). Torsins can protect the dopamine neurons of C. elegans from defined cellular stresses linked to dopamine dysfunction in a model for neurodegeneration associated with reactive oxygen species. Specifically, torsins can prevent neurodegeneration associated with reactive oxygen species induced by exposure to 6-OHDA. (Cao et al., J Neurosci, 2005, 25(1): 3801-3812).

Another model for scoring neurodegeneration in C. elegans is the transgenic C. elegans overexpressing cat-2, the worm homologue for human tyrosine hydroxylase (“TH”), an enzyme in the dopamine synthesis pathway. Overexpression of CAT-2 results in widespread loss of DA neurons (Cao et al., J Neurosci, 2005, 25(1): 3801-3812). Torsin proteins have been shown to have some neuroprotective actions on DA neurons. This model can be used for screening actions on TH-containing neurons such as adrenergic, noradrenergic, and DA neurons. Similar assays may be used to study the death and degeneration of different neuronal subtypes such as neurons containing serotonin, glutamate, GABA, glycine, acetylcholine, histamine, and peptide neurotransmitters.

A chemically diverse small molecule library was also screened to identify small molecule compounds with actions on preventing neurodegeneration associated with reactive oxygen species. The C. elegans small molecule library was obtained from Prestwick Chemical, Inc. (Washington, D.C.) (hereafter “Prestwick Library”). The library is a chemically diverse collection of 240 small molecules that have been selected for tolerability in C. elegans. All compounds in the library have been examined for toxicity over the lifetime of the worms and have been shown to be non-toxic for C. elegans. The candidate compounds do not color or obscure the incubation medium for histological studies. Over 95% of the compounds in the library are off-patent marketed drugs and have been safely administered to humans. Approximately 5% of these drugs are alkaloids. This library permits screening for drugs that provide hits with sufficient potency. If a positive result occurs but without sufficient potency, optimized analogs may be synthesized using computer-assisted drug design and readily screened in the same manner. The compounds in this library provide opportunities for further development since the initial lead is non-toxic, orally bioavailable with an acceptable half-life, and is well tolerated.

To facilitate the chances of success, compounds may be selected using computer programs such as ChemX/ChemDiverse (Accelrys) and based on critical devices by experienced medicinal chemists. To increase the diversity and increase the success of the screening process, compounds with known efficacy in different therapeutic areas may be assembled in this library. These compounds may include neuropsychiatry, anti-diabetic, antiviral, antihypertensive, antipyretic, anti-inflammatory, antibiotic, and anti-infective drugs.

Screening the small molecule library in a C. elegans model for neuroprotection has identified several classes of compounds with an effect on preventing neurodegeneration associated with reactive oxygen species. Drugs may be plated onto substrates where the transgenic worms are grown or administered in other conventional manners to expose the worms to the candidate drugs. When introduced into the growth substrate, the small molecule compounds penetrate the animals both by diffusion through the cuticle and ingestion. This mode of administration allows the continuous exposure of animals to the drug. When drugs produce a response at the initial screening concentration, serial dilutions were made to define the highest possible dose to cause the observed effect.

The following list is not intended to be limiting and provides specific small molecules with neuroprotective actions described herein.

I. Molecules that Assist the Function of Molecular Chaperones

The following classes of molecules prevent neurodegeneration and neuronal loss associated with protein misfolding and aggregation by modulating the function of molecular chaperones. The molecular chaperones primarily involved in regulating proper protein folding are the 40-kDa heat shock protein (HSP40; DnaJ), 60-kDa heat shock protein (HSP60; GroEL), 70-kDa heat shock protein (HSP70; DnaK), and Torsin (TOR-1; TOR-2; torsinA; torsinB; OOC-5) families. In one embodiment, these classes of molecules promote proper protein folding by modulating the actions of the torsinA protein.

a) Topoisomerase II Inhibitors

In one embodiment, topoisomerase II inhibitors are used for preventing neurodegeneration and neuronal loss associated with protein misfolding and aggregation. Topoisomerase II inhibitors may be selected from, but are not limited to, lomefloxacin, cinoxacin, amsacrine, etoposide, teniposide, oxoliic acid, nalidixic acid, suramin, merbarone, genistein, epirubicin HCl, ellipticine, doxorubicin, aurintricarboxylic acid (“ATA”) or pharmaceutically acceptable salts thereof.

Of particular importance is Nalidixic acid (NEGGRAM®, Sanofi-Aventis, Bridgewater, N.J.; CAS No. 3 89-08-2), which is a quinolone antibacterial agent generally known for treating infections of the urinary tract. Nalidixic acid belongs to the drug family of 4-quinolones which are quinolones containing a 4-oxo (a carbonyl in the para position to the nitrogen). They inhibit the A-subunit of DNA gyrase and are used as antimicrobials. Second generation 4-quinoloines are also substituted with a 1-piperazinyl group at the 7-position and a fluorine at the 6-position. As mentioned previously, the small molecule compounds included in the present invention have been approved for human use. The drug nalidixic acid is approved for use in the treatment of urinary tract infections and the recommended dosages are about 750 mg/kg to about 1500 mg/kg every 6 hours and more commonly is administered at about 1 gram/kg every 6 hours. If the medicine is taken for more than one or two weeks, the dosage may be decreased to about 500 mg/kg every 6 hours although this dosage can be titrated appropriately as needed. Peak serum levels of active drug average approximately 20 to 40 jig/mL (90% protein bound), 1 to 2 hours after administration of a 1 gram/kg dose to a fasting normal individual, with a half-life of about 90 minutes. This dosage of nalixic acid for preventing neurodegeneration and neuronal loss associated with protein misfolding and aggregation can be titrated appropriately as needed based on these effective and non-toxic doses for treating other disorders.

Another topoisomerase II inhibitor of particular importance is oxolinic acid (CAS No: 14698-29-4), an antibacterial agent used in the treatment of urinary tract infections. As mentioned previously, the small molecule compounds included the present invention have been approved for human use. The drug oxolinic acid is approved for use in the treatment of urinary tract infections and the recommended dosages are between about 10 mg/kg to about 40 mg/kg and more commonly at about 20 mg/kg. This dosage of oxolinic acid for preventing neurodegeneration and neuronal loss associated with protein misfolding and aggregation can be titrated appropriately as needed based on these effective and non-toxic doses for treating other disorders.

b) Bacterial Transpeptidase Inhibitors

In an alternative embodiment, bacterial transpeptidase inhibitors are used for preventing neurodegeneration and neuronal loss associated with protein misfolding and aggregation. Bacterial transpeptidase inhibitors may be selected from, but are not limited to, ampicillin, cloxacillin, piperacillin, amoxicillin, cefadroxil, dicloxyacillin, carbenicillin, penicillin, metampicillin, amoxicillin, cefoxatin or pharmaceutically acceptable salts thereof.

Of particular importance is the penicillin derivative metampicillin sodium salt (CAS No. 6489-97-0; Prestwick library compound 235). Metampicillin is commonly administered at between about 250 mg/kg and about 500 mg/kg every 8 hours. This dosage of metampicillin for preventing neurodegeneration and neuronal loss associated with protein misfolding and aggregation can be titrated appropriately as needed based on these effective and non-toxic doses for treating other disorders.

In one embodiment of the present invention, bacterial transpeptidase inhibitors are used for preventing neurodegeneration and neuronal loss associated with reactive oxygen species. Bacterial transpeptidase inhibitors may be selected from, but are not limited to, ampicillin, penicillin, pivampicillin, talampicillin, metampicillin, amoxicillin, and cefoxatin. In one embodiment, compounds that cross the blood brain barrier are used for preventing neurodegeneration and neuronal loss associated with reactive oxygen species.

Of particular importance is the penicillin derivative metampicillin sodium salt (CAS No. 6489-97-0; Prestwick library compound 235). As mentioned previously, the small molecule compounds included in the present method have been approved for human use. The drug metampicillin is approved for use in the treatment of bacterial infections and the recommended dosages are about 250 mg/kg and about 500 mg/kg every 8 hours, although this dosage can be titrated appropriately as needed. This dosage of metampicillin for providing neuroprotection can be titrated appropriately as needed based on these effective and non-toxic doses for treating other disorders.

II. Molecules that Exert their Action Through Reversing the Effects of Defective Chaperones.

The following classes of molecules prevent neurodegeneration and neuronal loss associated with protein misfolding and aggregation by reversing the actions of defective molecular chaperones. Included in this group are compounds that reverse the actions of torsin protein mutants with defective protein molecular chaperone activity.

a) Calcium Channel Antagonists

In one embodiment, calcium channel antagonists are used for preventing neurodegeneration and neuronal loss associated with protein misfolding and aggregation. Calcium channel antagonists may be selected from, but are not limited to, nimodipine, diproteverine, verapamil, nitrendipine, diltiazem, mioflazine, loperamide, flunarizine, bepridil, lidoflazine, CERM-196, R-58735, R-56865, ranolazine, nisoldipine, nicardipine, PN200-1 10, felodipine, amlodipine, R-(−)-202-791, or R-(+) Bay K-8644 or pharmaceutically acceptable salts thereof.

Of particular importance is loperaminde hydrochloride (IMODIUM®, McNeil-PPC, Inc., Fort Washington, Pa.; Mylan, CAS No. 53179-11-6) belongs to the group of opiate agonists and have widespread effects in the CNS and on smooth muscle due to activation of specific delta, mu, and kappa opiate receptors (each controlling different brain functions). As mentioned previously, the small molecule compounds that are included in the present invention have been approved for human use. The drug loperamide HCl is approved for use in the treatment of diarrhea and the recommended dosages are about 1 mg/kg to about 5 mg/kg initially with about 0.5 mg/kg to about 3 mg/kg afterwards and more commonly about 4 mg/kg initially with about 2 mg/kg afterwards not to exceed a daily dosage of about 16 mg/kg. This dosage of loperamide HCl for preventing neurodegeneration and neuronal loss associated with protein misfolding and aggregation can be titrated appropriately as needed based on these effective and non-toxic doses for treating other disorders.

b) Cyclooxygenase Inhibitors

In one embodiment, cyclooxygenase inhibitors are used to prevent neurodegeneration and neuronal loss associated with protein misfolding and aggregation. Cyclooxygenase inhibitors may be selected from, but are not limited to, naproxen, flufenamic acid, tolfenamic acid, fenbufen, ketoprofen, phenacetin, dipyrone, flurbiprofen, meclofenamide, piroxicam, indomethacine or pharmaceutically acceptable salts thereof. In addition to anti-inflammatory actions, cyclooxygenase inhibitors have analgesic, antipyretic, and platelet-inhibitory actions. They are used primarily in the treatment of chronic arthritic conditions and certain soft tissue disorders associated with pain and inflammation. Cyclooxygenase inhibitors include nonsteroidal anti-inflammatory drugs (“NSAIDs”) that act by blocking the synthesis of prostaglandins by inhibiting cyclooxygenase, which converts arachidonic acid to cyclic endoperoxides, precursors of prostaglandins. Inhibition of prostaglandin synthesis accounts for their analgesic, antipyretic, and platelet-inhibitory actions; other mechanisms may contribute to their anti-inflammatory effects. Certain NSAIDs also may inhibit lipoxygenase enzymes or phospholipase-C or may modulate T-cell function (AMA Drug Evaluations Annual, 1994, 1814-1815).

Of particular importance is meclofenamic acid sodium salt (Mylan, CAS No. 644-62-2). As mentioned previously, the small molecule compounds included in the present invention have been approved for human use. The drug meclofenamic acid sodium salt is approved for use in the treatment of pain and the recommended dosages are about 25 mg/kg to about 75 mg/kg and more commonly about 50 mg/kg 4 times/day but may be increased to about 400 mg/day. This dosage of meclofenamic acid sodium salt for preventing neurodegeneration and neuronal loss associated with protein misfolding and aggregation can be titrated appropriately as needed based on these effective and non-toxic doses for treating other disorders.

In one embodiment of the invention, cyclooxygenase inhibitors are used for preventing neurodegeneration and neuronal loss associated with reactive oxygen species. Cyclooxygenase inhibitors may be selected from, but are not limited to flurbiprofen, meclofenamide, piroxicam, and indomethacine. In addition to anti-inflammatory actions, they have analgesic, antipyretic, and platelet-inhibitory actions. They are used primarily in the treatment of chronic arthritic conditions and certain soft tissue disorders associated with pain and inflammation. NSAIDs act by blocking the synthesis of prostaglandins by inhibiting cyclooxygenase, which converts arachidonic acid to cyclic endoperoxides, precursors of prostaglandins. Inhibition of prostaglandin synthesis accounts for their analgesic, antipyretic, and platelet-inhibitory actions; other mechanisms may contribute to their anti-inflammatory effects. Certain NSAIDs also may inhibit lipoxygenase enzymes or phospholipase-C or may modulate T-cell function (AMA Drug Evaluations Annual, 1994, 1814-1815).

Of particular importance is meclofenamic acid sodium salt (Mylan Pharmaceuticals, Inc., CAS No. 644-62-2; Prestwick library compound 206). As mentioned previously, the small molecule compounds included in the present invention have been approved for human use. The drug meclofenamic acid is approved for the treatment of pain and the recommended dosages are about 25 mg/kg to about 75 mg/kg and more commonly about 50 mg/kg 4 times/day but may be increased to about 400 mg/day. This dosage of meclofenamic acid sodium salt for preventing neurodegeneration and neuronal loss associated with reactive oxygen species can be titrated appropriately as needed based on these effective and non-toxic doses for treating other disorders.

III. Molecules that Influence Polyglutamine Expansions

The following classes of molecules prevent neurodegeneration and neuronal loss associated with protein misfolding and aggregation by influencing aggregation-prone proteins. Included in these classes are molecules that influence proteins with polyglutamine repeats.

a) Folic Acid Synthesis Inhibitors

In one embodiment, folic acid synthesis inhibitors are used to prevent neurodegeneration and neuronal loss associated with protein misfolding and aggregation. Folic acid synthesis inhibitors may be selected from, but are not limited to, sulfonamides, including sulfamethoxazole, sulfadiazine, and sulfadoxine; dapsone; trimethoprim; diaveridine; pyrimethamine; methotrexate; or pharmaceutically acceptable salts thereof.

Of particular importance is mafenide (CAS No. 138-39-6; Prestwick library compound 166), a member of the sulfonamides that contains the structure SO₂NH₂. Members of this group, also known as “sulfa drugs,” are derivatives of sulfanilamide, which act as a folic acid synthesis inhibitors in microorganisms, and are bacteriostatic. As mentioned previously, the small molecule compounds included in the present invention have been approved for human use. The drug mafenide is approved for use as an anti-bacterial drug and the recommended dosages are about 500 mg/kg for the first dose, then about 250 mg/kg every six hours as needed for up to seven days. This dosage of mafenide for preventing neurodegeneration and neuronal loss associated with protein misfolding and aggregation can be titrated appropriately as needed based on these effective and non-toxic doses for treating other disorders.

b) Local Anaesthetics (Na⁺ Channel Blockers)

In one embodiment, sodium channel blockers are used to prevent neurodegeneration and neuronal loss associated with protein misfolding and aggregation. Sodium channel blockers may be selected from, but are not limited to, lidocaine, dyclonine HCl, mexilitine, phenyloin, ketamine, flecainide, amantadine or pharmaceutically acceptable salts thereof.

Of particular importance is dyclonine hydrochloride (DYCLONE®, AstraZeneca, DE, CAS No. 586-60-7). Dyclonine HCl is a local anesthetic agent that blocks nerve conduction when applied locally to nerve tissue in appropriate concentrations. Dyclonine acts on any part of the nervous system and on every type of nerve fiber. In contact with a nerve trunk, these anesthetics can cause both sensory and motor paralysis in the innervated area. Their action is completely reversible (From Gilman A G, et. al., Goodman and Gilman's The Pharmacological Basis of Therapeutics, 8th ed). Nearly all local anesthetics act by reducing the tendency of voltage-dependent sodium channels to activate. As mentioned previously, the small molecule compounds including the present invention have been approved for human use. The drug dyclonine HCl is approved for use as a local anesthetic and the recommended dosages are about 2-3 mg/kg every 2 hours. This dosage of dyclonine HCl for preventing neurodegeneration and neuronal loss associated with protein misfolding and aggregation can be titrated appropriately as needed based on these effective and non-toxic doses for treating other disorders.

In another embodiment of the invention, sodium channel blockers are used for preventing neurodegeneration and neuronal loss associated with reactive oxygen species. Sodium channel blockers may be selected from, but are not limited to, lidocaine, dyclonine HCl, mexilitine, phenyloin, ketamine, flecainide, and amantadine and are commonly used as local anesthetics. In contact with a nerve trunk, these anesthetics can cause both sensory and motor paralysis in the innervated area. Their action is completely reversible (From Gilman A G, et. al., Goodman and Gilman's The Pharmacological Basis of Therapeutics, 8th ed).

Of particular importance is lidocaine HCl (Alphacain HCl/anestacon/xylocalne, Astrazeneca, CAS No. 137-58-6; Prestwick Library compound 50). Lidocaine is a local anesthetic agent that blocks nerve conduction when applied locally to nerve tissue in appropriate concentrations. Lidocaine acts on any part of the nervous system and on every type of nerve fiber. As mentioned previously, the small molecule compounds included in the present invention have been approved for human use. Lidocaine is approved for use as a local anesthetic and the recommended dosages are about 1 mg/kg to about 50 mg/kg, more commonly about 5 mg/kg to about 35 mg/kg and most commonly about 10 mg/kg to about 20 mg/kg. This dosage of lidocaine HCl for preventing neurodegeneration and neuronal loss associated with reactive oxygen species can be titrated appropriately as needed based on these effective and non-toxic doses for treating other disorders.

TABLE 2
Compounds identified from a primary screen of the Prestwick
library with an effect on neurodegeneration and neuronal loss
associated with protein misfolding and aggregation

| Prestwick | | | |
|---|---|---|---|
| library | | | Specificity |
| compound | Chemical name | Known Function | in worm |
| 166 | Mafenide | Antibacterial; | assay |
| | | inhibitor of folic | Polyglutamine |
| | | acid_synthesis | expansion diseases |
| 264 | Dyclonine HCl | Local anaesthetic; | Polyglutamine |
| | (DYCLONE ®) | Na⁺ channel | expansion diseases |
| | | blocker | |
| 187 | Nalidixic Acid | Antibacterial; | wt torsinA |
| | (NEGGRAM ®) | topoisomerase II | |
| | | inhibitor | |
| 193 | Oxolinic Acid | Antibacterial; | wt torsinA |
| | | topoisomerase II | |
| | | inhibitor | |
| 235 | Metampicillin | Antibacterial; | wt torsinA |
| | sodium salt | bacterial | |
| | | transpeptidase | |
| | | inhibitor | |
| 144 | Loperamide HCl | Anti-diarrheal; | Mutant torsinA |
| | (MODIUM ®) | Ca2+ channel | (ΔE) |
| | | antagonist | |
| 206 | Meclofenamic | Anti- | Mutant torsinA |
| | acid | inflammatory; | (ΔE) |
| | sodium salt | cyclooxygenase | |
| | | inhibitor | |

Quantitative Structure-Activity Relationship (“QSAR”) methods may be used to quantify the relationship between the chemical structure of a compound and its biological activity. Each compound class may be quantified or rated for broad-spectrum efficacy using one or more techniques that includes a structure-activity relationship (“SAR”) and/or a QSAR method which identify one or more activity related to one or more structures that are related to the class of compounds. Each of these compound classes may then be prioritized based on such factors as synthesizability, flexibility, patentability, activities, toxicities, and/or metabolism. In this case, all or an additional set of compounds within each particular compound class may be assayed and analyzed. As some compound classes may be very large, a subset of the compounds in the classes may be assayed and analyzed and if the class continues to demonstrate efficacy in excess of a predetermined level, the remaining members will be assayed. This approach will also identify functional analogues of compounds and classes of compounds for use in the present methods. The activity of functional analogues may be confirmed using the C. elegans model to screen for protein aggregation.

In addition to the compounds described above as having particular importance, other related chemical compounds contained in the Prestwick library have been identified within the chemical classes described above. A list of these compounds is provided in Table 3.

TABLE 3
Related compounds from Prestwick library

| Subject/Target | |
|---|---|
| Compound | Related compounds from Prestwick library |
| Topoisomerase II | 238: LomeIloxacin hydrochloride; C₁₇H₂₀C1F₂N₃O₃ |
| inhibitors | 780: Cinoxacin; C₁₂H₁₀N₂O₅ |
| Bacterial | 114: ampicillin sodium salt; C₁₆H₁₈N₃NaO₄S |
| Transpeptidase | 186: Cloxacillin sodium salt; C₁₉H₁₇C1N₃NaO₅S |
| Inhibitors | 755: Piperacillin sodium salt; C₂₃H₂₆N₅NaO₇S |
| | 357: Amoxiciflin; C₁₆H₁₉N₃O₅S |
| | 434: Cefadroxil; C₁₆H₁₇N₃O₅S |
| | 450: Dicloxyacillin sodium salt; C₁₉H₁₆C1₂N₃NaO₅5 |
| | 703: Carbenicillin disodium salt; C₁₇H₁₆N₂Na₂O₆5 |
| Calcium Channel | 134: Diltiazem hydrochloride; C₂₂H₂₇C1N₂O₄S |
| Inhibitors | |
| Cyclooxygenase | 45: Naproxen; C₁₄H₁₄O₃ |
| Inhibitors | 203: Flufenamic acid; C₁₄H₁₀F₃NO₂ |
| | 205: Tolfenamic acid; C₁₄H₁₂C1NO₂ |
| | 218: Fenbufen; C₁₆H₁₄O₃ |
| | 219: Ketoprofen; C₁₆H₁₄O₃ |
| | 533: Phenacetin; C₁₀H₁₃NO₂ |
| | 713: Dipyrone; C₁₃H₁₆N₃NaO₄S |
| Folic Acid | 14: Sulfacetamide sodic hydrate; C₈H₁₁N₂NaO₄S |
| Synthesis | 23: Sulfadiazine; C₁₀H₁₀N₄O₂S |
| Inhibitors | 10: Sulfaguanidine; C₇H₁₀N₄O₂S |
| | 16: Sulfathiazole; C₉H₉N₃O₂S₂ |
| | 177: Sulfamethoxazole; C₁₀H₁₁N₃O₃S |
| | 711: Sulfabenzamide; C₁₃H₁₂N₂O₃S |
| Sodium Channel | 264: Dyclonine hydrochloride; C₁₈H₂₈ClNO₂ |
| Inhibitors | 49: Amyleine HCl; C₁₄H₂₂C1NO₂ |
| | 76: Dibucaine; C₂₀H₂₉N₃O₂ |
| | 312: Flunarizine dihydrochloride; C₂₆H₂₃C1₂F₂N₂ |
| | 41: Procaine HCl; C₁₃H₂₁C1N₂O₂ |
| | 199: Prilocaine HCl; C₁₃H₂₁C1N₂O |
| | 241: Mexiletine HCl; C₁₁H₁₈C1NO |
| | 266: Disopyramid; C₂₁H₂₉N₃O |
| | 409: Amiodarone HCl; C₂₅H₃₀C1I₂NO₃ |
| | 58: Oxethazaine; C₂₈H₄₁N₃NO₃ |
| | 305: Bupivacaine HCl; C₁₈H₂₉C1N₂0 |
| | 57: Benoxinate HCl; C₁₇H₂₉C1N₂O |

The compounds listed in Table 3 were subjected to a primary screening assay in C. elegans. A number of compounds were found to prevent neurodegeneration and neuronal loss associated with protein misfolding and aggregation despite not having significant actions in the preliminary screening process. This data is presented in Example 1. Other in vitro and in vivo screening assays are known in the art for screening these drugs to confirm the results from the preliminary and secondary screens. A negative result from the preliminary screen may result in a positive effect using a different assay. Other assays of protein misfolding and aggregation include the dynamic light scattering assay (L1 et al., FASEB J., 2004, ePub; Kaylor et al., J Mol Biol, 2005, 353: 357-372), sedimentation velocity analysis (MacRaild et al., J Biol Chem, 2004, 2779: 21038-21045) and yeast aggregation assay (Outeiro et al., Science, 2003, 302: 1772)—although other assays are known in the art and may be used for these purposes.

Similarly, related chemical compounds and functional analogues within the specified drug classes or those compounds identified using QSAR may also be screened using any of these protein misfolding/aggregation assays to determine the activity on preventing neurodegeneration and neuronal loss associated with protein misfolding and aggregation. High throughput screening techniques may be used to screen variants of the drugs identified in the primary C. elegans screen for an effect on preventing neurodegeneration and neuronal loss associated with protein misfolding and aggregation. Computer-assisted drug design/computer modeling methods may also be used to identify chemical variants that may be screened for actions on neurodegeneration and neuronal loss associated with protein misfolding and aggregation.

Computer modeling technology allows visualization of the three-dimensional atomic structure of a selected molecule and the rational design of new compounds that will interact with the molecule. These methods provide a way to find functional analogues of small molecule compounds that are known to have actions on neurodegeneration and neuronal loss associated with protein misfolding and aggregation. Analysis of the three dimensional structure of a compound as it binds to a target protein will identify the site of interaction which is then used to identify similar compounds and functional analogues that would have similar binding properties. The three-dimensional construct typically depends on data from x-ray crystallographic analyses or NIVIR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

In addition, some of the compounds listed in Table 3 were subjected to a primary screening assay in C. elegans. A number of compounds were found to prevent neurodegeneration and neuronal loss associated with reactive oxygen species despite not having significant actions in the preliminary screening process. This data is presented in Example 2. Other in vitro and in vivo screening assays are known in the art for screening these drugs to confirm the results from the preliminary and secondary screens. A negative result from the preliminary screen may result in a positive effect using a different assay for neuroprotection.

Similarly, related chemical compounds and functional analogues within the specified drug classes or those compounds identified using QSAR may also be screened using any assay of neurodegeneration to determine the activity for preventing neurodegeneration and neuronal loss associated with reactive oxygen species. Such assays are known to those of skill in the art and include in vivo and in vitro assays, including cell culture assays and transgenic animal models of neurodegeneration. High throughput screening techniques may be used to screen variants of the drugs identified in the primary C. elegans screen for an effect on neurodegeneration. Computer-assisted drug design/computer modeling methods may also be used to identify chemical variants that may be screened for actions on neurodegeneration and neuronal loss associated with reactive oxygen species.

Computer modeling technology allows visualization of the three-dimensional atomic structure of a selected molecule and the rational design of new compounds that will interact with the molecule. These methods provide a way to find functional analogues of known small molecule compounds that are known to have actions on neurodegeneration and neuronal loss associated with reactive oxygen species. Analysis of the three dimensional structure of a compound as it binds to a target protein will identify the site of interaction which is then used to identify similar compounds and functional analogues that would have similar binding properties. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling, and analysis of molecular structure. QUANTA allows interactive construction, modification, visualization, and analysis of the behavior of molecules with each other.

A number of articles review computer modeling of drugs interactive with specific proteins (Schneider and Fechner, Nat Rev Drug Discov, 2005 August, 4(8): 649-663; Guner, IDrugs, 2005 July, 8(7): 567-572; and Hanai, Curr Med Chem, 2005, 12(5): 501-525). Other computer programs that screen and graphically depict chemicals are available from companies such as BioDesign, Inc., Pasadena, Calif., and Hypercube, Inc., Cambridge, Ontario. Although these are primarily designed for application to drugs specific to particular proteins, they can be adapted to design of drugs specific to regions of DNA or RNA, once that region is identified. Although described above with reference to design and generation of compounds that could alter binding, one could also screen libraries of known compounds, including natural products or synthetic chemicals, and biologically active materials, including proteins, for compounds that are inhibitors or activators. The activity of compounds identified using this approach may be confirmed using the C. elegans model to screen for protein aggregation or neuroprotection.

In another aspect of the invention, the small molecule compounds work through torsin-dependent mechanisms to prevent neurodegeneration and neuronal loss associated with reactive oxygen species. Small molecule compounds may be identified using the methods described herein that have actions on modulating the actions of torsin proteins to protect neurons from damage associated with reactive oxygen species. The compounds may modulate the actions of torsin proteins through direct or indirect interactions. Indirect actions may comprise modulating another enzyme or chemical intermediate that would have downstream actions on torsin proteins. In one embodiment, the compound modulating the actions of torsin proteins comprises metampicillin or other bacterial transpeptidase inhibitors.

In some embodiments of the invention, the composition may further comprise at least one reactive oxygen species scavenger. Suitable reactive oxygen species scavengers include coenzyme Q, vitamin E, vitamin C, pyruvate, melatonin, niacinamide, N-acetylcysteine, glutathione (“GSH”), and nitrones. In some embodiments, at least one reactive oxygen species scavenger is administered prophylactically in combination with the prophylactic administration of the small molecule compound.

The compounds useful in the present methods, or pharmaceutically acceptable salts thereof, can be delivered to a patient using a wide variety of routes or modes of administration. Suitable routes of administration include, but are not limited to, inhalation, transdermal, oral, rectal, transmucosal, intestinal, and parenteral administration—including intramuscular, subcutaneous, and intravenous injections.

The term “pharmaceutically acceptable salt” means those salts which retain the biological effectiveness and properties of the compounds used in the present methods, and which are not biologically or otherwise undesirable. Such salts may be prepared from inorganic and organic bases. Salts derived from inorganic bases include, but are not limited to, the sodium, potassium, lithium, ammonium, calcium, and magnesium salts. Salts derived from organic bases include, but are not limited to, salts of primary, secondary, and tertiary amines, substituted amines including naturally-occurring substituted amines, and cyclic amines, including isopropylamine, trimethylamine, diethylamine, triethylamine, tripropylamine, ethanolamine, 2-dimethylaminoethanol, tromethanine, lysine, arginine, histidine, caffeine, procaine, hydrabamine, choline, betaine, ethylenediamine, glucosamine, N-alkylglucamines, theobromine, purines, piperazine, piperidine, and N-ethylpiperidine. It should also be understood that other carboxylic acid derivatives would be useful in the practice of this method, such as carboxylic acid amides, including carboxamides, lower alkyl carboxamides, di(lower alkyl) carboxamides, and the like.

The compounds, or pharmaceutically acceptable salts thereof, may be administered singly, in combination with other compounds, and/or in cocktails combined with other therapeutic agents. Of course, the choice of therapeutic agents that can be co-administered with the compounds of the present method will depend, in part, on the condition being treated.

The active compounds (or pharmaceutically acceptable salts thereof) may be administered per se or in the form of a pharmaceutical composition wherein the active compound(s) is in admixture or mixture with one or more pharmaceutically acceptable carriers, excipients, or diluents. Pharmaceutical compositions for use in accordance with the present invention may be formulated in conventional manner using one or more physiologically acceptable carriers including excipients and auxiliaries which facilitate processing of the active compounds into preparations which can be used pharmaceutically. Proper formulation is dependent upon the route of administration chosen.

For injection, the compounds may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compound(s) with pharmaceutically acceptable carriers well known in the art. Such carriers enable the compounds of the present method to be formulated as tablets, pills, dragees, capsules, liquids, gels, syrups, slurries, suspensions, and the like, for oral ingestion by a patient to be treated. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (“PVP”). If desired, disintegrating agents may be added, such as the cross-linked PVP, agar, alginic acid, or a salt thereof such as sodium alginate.

Dragee cores can be provided with suitable coatings. For this purpose, concentrated sugar solutions may be used, which may optionally contain gum arabic, talc, PVP, carbopol gel, polyethylene glycol (“PEG”), and/or titanium dioxide, lacquer solutions, and suitable organic solvents or solvent mixtures. Dyestuffs or pigments may be added to the tablets or dragee coatings for identification or to characterize different combinations of active compound doses.

For administration orally, the compounds may be formulated as a sustained release preparation. Numerous techniques for formulating sustained release preparations are described in the following references—U.S. Pat. Nos. 4,891,223; 6,004,582; 5,397,574; 5,419,917; 5,458,005; 5,458,887; 5,458,888; 5,472,708; 6,106,862; 6,103,263; 6,099,862; 6,099,859; 6,096,340; 6,077,541; 5,916,595; 5,837,379; 5,834,023; 5,885,616; 5,456,921; 5,603,956; 5,512,297; 5,399,362; 5,399,359; 5,399,358; 5,725,883; 5,773,025; 6,110,498; 5,952,004; 5,912,013; 5,897,876; 5,824,638; 5,464,633; 5,422,123; and 4,839,177; and WO 98/47491.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid PEG. In addition, stabilizers may be added. All formulations for oral administration should be in dosages suitable for such administration.

For buccal administration, the compositions may take the form of tablets or lozenges formulated in conventional manner.

For administration by inhalation, the active compound(s) may be conveniently delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide, or other suitable gas. In the case of a pressurized aerosol the dosage unit may be determined by providing a valve to deliver a metered amount. Capsules and cartridges of compounds such as gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compound(s) may be in powder form for constitution with a suitable vehicle, such as sterile pyrogen-free water, before use.

The compounds may also be formulated in rectal compositions such as suppositories or retention enemas, such as compounds containing conventional suppository bases such as cocoa butter or other glycerides.

In addition to the formulations described previously, the compounds may also be formulated as a depot preparation. Such long acting formulations may be administered by implantation or transcutaneous delivery (for example subcutaneously or intramuscularly), intramuscular injection, or a transdermal patch. Thus, for example, the compounds may be formulated with suitable polymeric or hydrophobic materials (for example, as an emulsion in an acceptable oil) or ion exchange resins, or as sparingly soluble derivatives such as a sparingly soluble salt.

A further embodiment of the present invention is related to a nanoparticle. The compounds described herein may be incorporated into the nanoparticle. The nanoparticle within the scope of the invention is meant to include particles at the single molecule level as well as those aggregates of particles that exhibit microscopic properties. Methods of using and making the above-mentioned nanoparticle can be found in the art (U.S. Pat. Nos. 6,395,253; 6,387,329; 6,383,500; 6,361,944; 6,350,515; 6,333,051; 6,323,989; 6,316,029; 6,312,731; 6,306,610; 6,288,040; 6,272,262; 6,268,222; 6,265,546; 6,262,129; 6,262,032; 6,248,724; 6,217,912; 6,217,901; 6,217,864; 6,214,560; 6,187,559; 6,180,415; 6,159,445; 6,149,868; 6,121,005; 6,086,881; 6,007,845; 6,002,817; 5,985,353; 5,981,467; 5,962,566; 5,925,564; 5,904,936; 5,856,435; 5,792,751; 5,789,375; 5,770,580; 5,756,264; 5,705,585; 5,702,727; and 5,686,113).

The pharmaceutical compositions also may comprise suitable solid or gel phase carriers or excipients. Examples of such carriers or excipients include, but are not limited to, calcium carbonate, calcium phosphate, various sugars, starches, cellulose derivatives, gelatin, and polymers such as PEG.

Pharmaceutical compositions suitable for use in the present methods include compositions wherein the active ingredient is contained in a therapeutically or prophylactically effective amount, i.e., in an amount effective to achieve therapeutic or prophylactic benefit, as previously discussed. Of course, the actual amount effective for a particular application will depend, inter alia, on the condition being treated and the route of administration. Determination of an effective amount is well within the capabilities of those skilled in the art, especially in light of the disclosure herein.

Therapeutically effective amounts for use in humans can be determined from animal models. For example, a dose for humans can be formulated to achieve a circulating concentration that has been found to be effective in animals. Useful animal models of pain are well known in the art.

One skilled in the art, without undue experimentation, can devise a dosing strategy—a combination of dose level and dose frequency—which will result in substantially continuous maintenance of the plasma level of the small molecule compounds within the desired concentration range for the specified period of time in each dosing period and, therefore, maximize the desired neuroprotection. Continuous exposure can be achieved by the use of sustained release drug delivery systems, including implanted or parenteral polymers or slow-release or pulse-release oral formulations. It is also well known to those skilled in the art that maintaining plasma exposure over a threshold level can also be achieved by matching a drug/formulation combination to a dose level and dosing schedule. These procedures are known in the field by various names, including “dosing up” or “dosing to steady state.” As an example, an oral formulation which results in a short half-life of drug levels in plasma can be dosed at a higher level or dosed more frequently to maintain plasma levels above a desired threshold—such dose and dosing schedule chosen based on mathematical modeling of the pharmacokinetic profile of the formulation using published formulas or calculations or commercially available software programs known to those skilled in the art. As another example, a formulation which results in extended exposure at high levels can be dosed on a less frequent schedule, such dose and dosing schedule chosen based on mathematical modeling of the pharmacokinetic profile of the formulation using published formulas or calculations or commercially available software programs known to those skilled in the art. As another example, a formulation which results in extended exposure at low levels can be “dosed up” using a more frequent dosing schedule until the plasma levels are shown to be or are predicted to be within the defined range, such dose and dosing schedule chosen based on mathematical modeling of the pharmacokinetic profile of the formulation using published formulas or calculations or commercially available software programs known to those skilled in the art.

The compositions of the present invention may be administered prophylactically to an individual at risk for CNS injury of for developing a neurodegenerative disease. In another embodiment, the compositions of the present invention may be administered to an individual after a positive test result from genetic screening for a neurodegenerative disorder when the individual is still asymptomatic, or else at the onset of the disease when clinical symptoms of a neurodegenerative disease begin to manifest. The compositions of the present method may be administered for the duration of a time period where the individual would be at risk for CNS injury or for developing a neurodegenerative disease. In any of the methods described herein, the compositions described inter alia may be administered at an effective amount to prevent neurodegeneration.

In another embodiment, the compositions of the present invention are administered to an individual immediately after suffering a traumatic brain injury or ischemic insult, such as a stroke, where factors secondary to the injury or ischemic insult—such as the formation of reactive oxygen species—may result in secondary neuronal damage. The compounds may be administered for at least about 3, 9, 18, or 24 hours after the injury or ischemic insult or also for at least about 3, 5, 7, 12, or 15 days after the injury or ischemic insult. Longer time periods of administration lasting at least one month may also be used depending on the degree of the injury.

In any of the methods of the present invention, the compositions described herein may be administered at an effective amount to treat or prevent neurodegeneration and neuronal loss. The compounds may also be administered at an effective amount to treat or prevent neurodegeneration and neuronal loss due to secondary damage from a CNS injury or ischemic insult. The compounds may be administered for a period of time sufficient to ameliorate or alleviate symptoms of the CNS injury or neurodegenerative disease. Various neuronal subtypes may be protected by the small molecule compounds described herein. Such neuronal subtypes include, but are not limited to, adrenergic, noradrenergic, serotonergic, dopaminergic, cholinergic, GABAergic, glycinergic, glutamatergic, and histaminergic neurons.

In another embodiment, the compounds described herein may be administered to modulate the activity of molecular chaperone proteins such as those within the torsin family of proteins. These methods have particular relevance to disorders where molecular chaperone activity is impaired or exacerbated. Of particular importance is the administration of compounds for treating early-onset torsin dystonia where a mutation in the torsinA protein impairs molecular chaperone activity and is responsible, in part, for the neuronal dysfunction associated with dystonia.

The compounds may also be administered to modulate the activity of neurotransmitter transporters. Such transporters may include, but are not limited to, the dopamine transporter (“DAT”), the serotonin transporter, the GABA transporter, the noradrenaline transporter, the vesicular acetylcholine transporter, and the like. Modulation of torsin proteins and neurotransmitter transporters may be used to provide neuroprotection to neurons at risk of damage or death.

In another aspect, the small molecule compounds work through torsin dependent mechanisms to treat or prevent neurodegeneration and neuronal loss associated with protein misfolding or aggregation. Small molecule compounds may be identified using the methods described herein that have actions on modulating the actions of torsin proteins to treat or prevent neurodegeneration and neuronal loss associated with protein misfolding or aggregation. The compounds may modulate the actions of torsin proteins through direct or indirect interactions. Indirect actions may comprise modulating another enzyme or chemical intermediate that would have downstream actions on torsin proteins. In one embodiment, the compound modulating the actions of torsin proteins comprises metampicillin or other bacterial transpeptidase inhibitors. In another embodiment, the compound modulating the actions of torsin proteins comprises nalidixic acid or oxolinic acid or other topoisomerase II inhibitors. In yet another embodiment, the compounds modulate mutant torsin proteins for treating or preventing neurodegeneration and neuronal loss associated with protein misfolding or aggregation. In this particular embodiment, the compounds comprise calcium channel antagonists, such as loperamide HCl, or cyclooxygenase inhibitors, such as meclofenamic acid sodium salt monohydrate.

The following examples will serve to further illustrate the present invention without, at the same time, constituting any limitation thereof. It is to be clearly understood that resort may be had to various embodiments, modifications, and equivalents thereof which, after reading the description herein, may suggest themselves to those skilled in the art without departing from the spirit of the invention.

### EXAMPLES

### Example 1

### Screening a Small Molecule Library Using C. elegans Models of Protein Misfolding and Aggregation

C elegans nematodes were grown at 20° C. on NGM plates as described by Brenner (Brenner, Genetics, 1974, 77: 71-94). Several transgenic C. elegans lines were used for primary screens of the Prestwick small molecule library. A transgenic worm line expressing P_{unc-54}::Q82-GFP; with both wild type (“wt”) (P_{unc=54}: :torsinA) or mutant (P_{unc-54}::torsinA(ΔE)). Torsin-A expresses a phenotype that results in visible protein aggregation under fluorescent microscope. Transgenic worms were plated on drug plates and progeny were studied for a return to soluble protein.

Drugs were administered to C. elegans according to a standard procedure (Rand and Johnson, Methods Cell Biol, 1995, 48: 187-204), by mixing the solubilized drug with the agar medium on which the worms are grown. This mode of administration allows the continuous exposure of worms to the drug.

Each drug was first dissolved in an appropriate solvent, followed by adding the drug solution into pre-autoclaved media, with the volume of drug solution already taken into account. All drugs were tested at 0.5 mg/ml initial concentration, a few of which were toxic to worms and then tested at 0.1 mg/ml or 0.025 mg/ml concentration. Each plate was seeded with 100 μl concentrated E. coli OP50 bacteria.

The screening assays used to identify the compounds use protein aggregation readouts to determine the specificities of molecular action. The screen took advantage of cellular assays in the animal model system, C. elegans using polyglutamine expansions. Additionally torsinA was included in some of the assays in 3 different ways: the presence of wild type torsinA, the presence of mutant torsinA, or the presence of both wild type and mutant torsinA (in all cases with polyglutamine expansions). Worms were screened for statistically significant reduction in protein aggregation. Compounds that yielded positive results were rescreened and differentiated based on the assay results.

The methods for the aggregate analysis assay are described in Caldwell et al. (Hum Mol Genetics, 2003, 12(3): 307-319). Briefly, worms were examined using a Nikon Eclipse E800 epifluorescence microscope equipped with Endow GFP HYQ and Texas Red HYQ filter cubes (Chroma Inc.). Images were captured with a Spot RI CCD camera (Diagnostic Instruments Inc.). MetaMorph Software (Universal Imaging Inc.) was used for pseudocoloration of images, image overlay, and aggregate size quantitation. For each worm line analyzed, average aggregate size was determined by capturing images of all aggregates in the posterior region of 30 L3-staged animals (for Q82::GFP aggregates) or all aggregates in 30 adult animals/day (for Q19::GFP analyses) at 1000× magnification. Pixel area was converted to μm in the MetaMorph software system and was directly downloaded to Excel spreadsheets for further analysis. Statistical analysis of aggregate size was performed by ANOVA using Statistica (SPPS Software).

To see the effect of the candidate compounds on suppressing protein misfolding and aggregation, five gravid adult worms were placed onto each drug plate and grown at 20° C. for approximately 3 days. Thirty Fl worms at L3 stage and young adult stage were counted for aggregate number.

To see the preventative effects of the candidate drug compounds on protein misfolding and aggregation, five gravid adult worms were placed onto each drug plate and grown at 20° C. for approximately 3 days. 35-40 Fl worms at L2 stage were picked and transferred to a control plate with the same concentration of control solvent compared to the drug plate. Thirty worms at L3 stage were counted for aggregate number 8 hours later. Thirty young adults were analyzed 32 hours later.

To calculate the scaled effectiveness of special regimen of drugs on worms based on standardized decrease in protein aggregates. The following formula was used:
(A−B)/(A−C)×100%, where

A=number of aggregates for worms growing on solvent control plates

B=number of aggregates for worms grown on the specific drug plate for the entire life of the worms

C=number of aggregates for worms that received pre-L2 exposure and were then removed to solvent control plates

If the regimen has no effect, the value would equal 0%. Conversely, if the regimen was as effective as raising the worms on the drug for their entire life, the value would equal 100%.

To see the corrective effect of the candidate drug compounds on protein misfolding and aggregation, five gravid adult worms were placed onto a solvent control plate and grown at 20° C. for approximately 3 days. 35-40 Fl worms at L2 stage were picked and transferred to a drug plate. Thirty worms at L3 stage were counted for aggregate number 8 hours later. Thirty young adults were analyzed 32 hours later.

To calculate the scaled effectiveness of special regimen of drugs on worms based on standardized decrease in protein aggregates. The following formula was used:
(A−B)/(A−C)×100%, where

A=number of aggregates for worms growing on solvent control plates

B=number of aggregates for worms grown on the specific drug plate for the entire life of the worms

C=number of aggregates for worms that received exposure from L2 stage (until adulthood)

If the regimen has no effect, the value would equal 0%. Conversely, if the regimen was as effective as raising the worms on the drug for their entire life, the value would equal 100%.

Results

Primary screening of the library identified 7 molecules that reproducibly reduce protein aggregates in worms containing P_{unc-54}::Q82::GFP+P_{unc-54}::torsinA+P_{unc54}::torsinA (ΔE).

To elucidate the mechanism by which the small molecule compounds act, all drugs identified in the primary screen were used to treat transgenic worms containing the transgene P_{unc-54}::Q82::GFP without any torsinA expression. Of the seven compounds identified in primary screen, 2 drugs, mafenide and dyclonine hydrochloride, reduced the presence of protein aggregates. This indicates that these two drugs prevent protein misfolding and aggregation through a torsin-independent mechanism likely acting directly on the polygutamine protein.

The remaining five primary candidate drugs that did not act directly on polyglutamine alone were plated in the presence of worms expressing the transgene P−_{unc.54}::Q82::GFP+P_{unc-54}::torsinA with wt torsinA only. Three of the drugs identified from the primary screen, nalidixic acid, oxolinic acid and metampicillin sodium salt reduced protein aggregation through wild type torsinA.

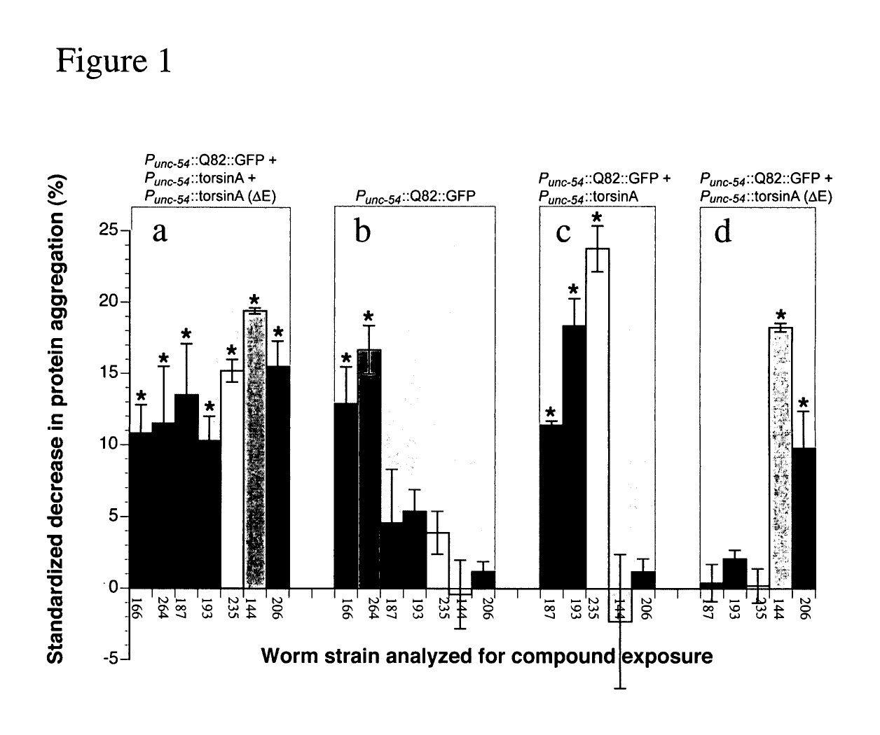

The same five primary candidate drugs that did not act directly on polyglutamine alone were also plated in the presence of worms expressing the transgene P_{unc-54}::Q82::GFP+P unc-54::torsinA (ΔE) with a mutant torsinA only. Two of the drugs, loperamide hydrochloride and meclofenamic acid sodium salt, reduced aggregation by acting on the mutant torsinA protein. These data are collectively shown in FIG. 1.

Molecules that have similar structures and mechanism of action to the 5 molecules acting through torsin-dependent mechanisms were re-analyzed for protein aggregation suppression. These molecules did not pass the first round of analysis for potential therapeutics and are listed in Table 2 above.

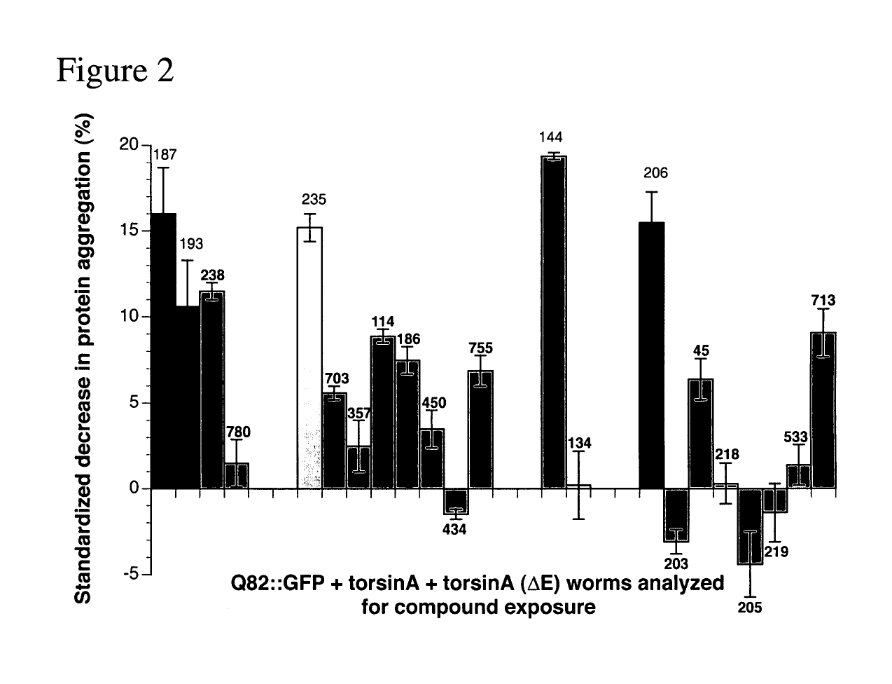

All molecules are coded by Prestwick Library number. Screening of related drugs in the C. elegans aggregate model resulted in variable actions on protein aggregate formation. These data are shown in (FIG. 2). While some of the compounds tested did not demonstrate a significant action on protein misfolding in this model, it is possible that other protein misfolding/aggregation assays may demonstrate actions on the formation of protein aggregates. Experiments showing positive response by the drug candidates were repeated multiple times to confirm the actions on protein misfolding and aggregation.

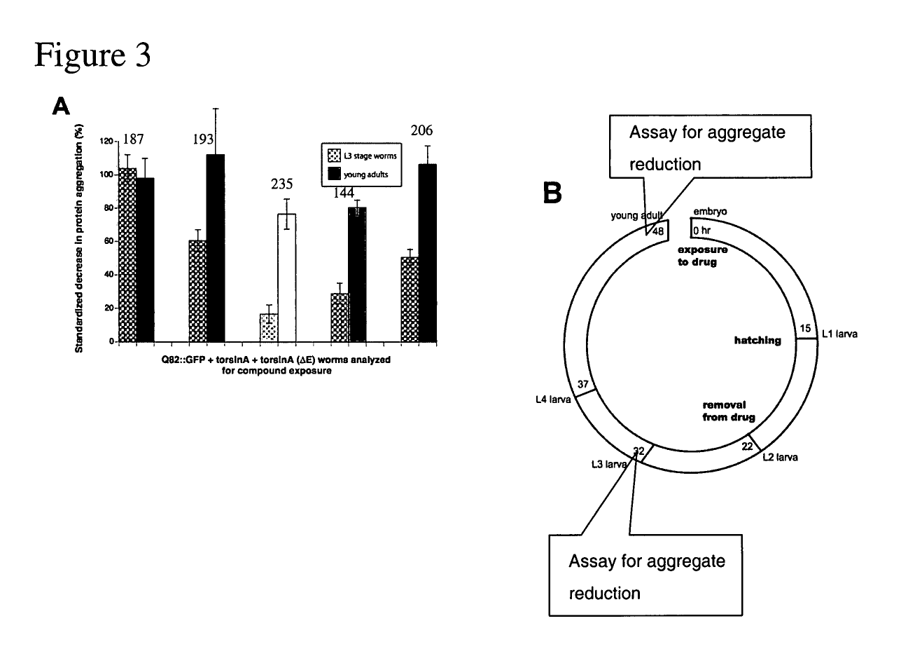

Results from the prevention assays show that all 5 torsin-specific drugs (nalidixic acid, oxolinic acid, metampicillin sodium salt, Loperamide HCl, Meclofenamic acid sodium salt) seem to be beneficial when the worms are exposed to the drug compound from hatching to L2 stage. Notably, the longer the worms age (despite the lack of exposure), the greater the effectiveness of the drugs (solid bars on graph). These results are shown in FIG. 3.

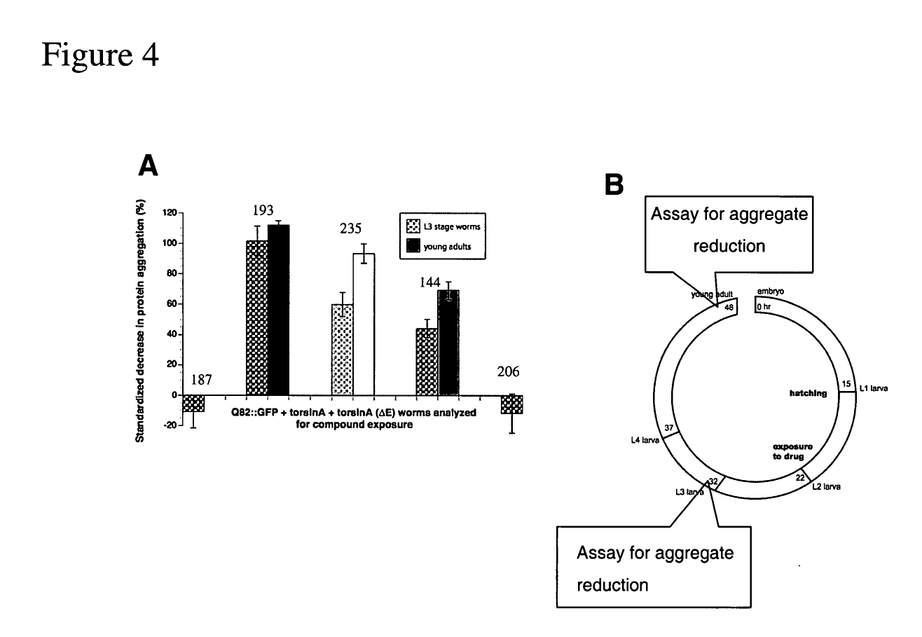

Results from the corrective assays show that 3 torsin-specific drugs (oxolinic acid, metampicillin sodium salt, and Loperamide HCl) seem to be beneficial when the drug compound is not provided until later in development. These results are shown in FIG. 4.

These results demonstrate that a library of candidate drug compounds may be screened in this C. elegans model for an effect on preventing or correcting protein misfolding and aggregation. Broader classes of these drugs may also be screened using this model to identify other drug compounds that have an effect on preventing or correcting protein misfolding and aggregation.

### Example 2

### Neuroprotection of Dopaminergic Neurons in C. elegans by Compounds Identified from the Prestwick Small Molecule Library

Previous studies have established that the “CEP” and “ADE” mechanosensory neurons in C. elegans undergo readily discernable neuronal degeneration after treatment with the dopamine-selective neurotoxin 6-OHDA (Nass et al, 2002). The toxicity of 6-OHDA is mediated through the formation of reactive oxygen species by the generation of hydrogen peroxide and hydroxide radicals via a nonenzymatic auto-oxidation process (Kumar et al., 1995; Foley and Riederer, 2000). After exposure to 6-OHDA, C. elegans dopamine neurons exhibit a characteristic dose dependent pattern of apoptotic cell death that was confirmed by ultrastructural analysis (Nass et al., 2002). This degeneration can be monitored in living animals by coexpressing with green fluorescent protein and categorized into three temporally and morphologically distinct stages, including neuronal process blebbing, cell body rounding with process loss, and cell body loss. These characteristic changes reproducibly appear in this order within a few hours, recapitulating observations in MPTP-treated monkeys and in 6-OHDA-treated rats, in which damage to striatal terminals leads to retrograde changes and precedes that of SNpc cell bodies (Berger et al., 1991; Herkenham et al., 1991). The actions of torsin protein in 6-OHDA-mediated neurodegeneration has recently been shown (Cao et al., J Neurosci, 25(1):3801-3812). Animals were treated with various concentrations of 6-OHDA and neurodegeneration was followed over time, as worms develop and age, by co-expression of a dopamine GFP marker in these transparent animals.

C. elegans nematodes were grown at 25° C. on NGM plates as described by Brenner (Brenner, Genetics, 1974, 77: 71-94). Two transgenic C. elegans lines were used for primary screens of the Prestwick small molecule library. A transgenic worm line expressing P_{dat-1}::GFP; with both wild type (P_{dat-1}::torsinA) or mutant (P_{dat-1}:::torsinA(ΔE)). Torsin-A expresses a phenotype that results in visible neurodegeneration after treatment with 6-OHDA. Transgenic worms were plated on drug plates and progeny were studied for morphological changes in the 8 dopaminergic neurons present in C. elegans.

Drugs were administered to C. elegans according to a standard procedure (Rand and Johnson, Methods Cell Biol, 1995, 48: 187-204) by mixing the solubilized drug with the agar medium on which the worms are grown. This mode of administration allows the continuous exposure of worms to the drug.

Each drug was first dissolved in an appropriate solvent, followed by adding the drug solution into pre-autoclaved media, with the volume of drug solution already taken into account. All drugs were tested at 0.5 mg/ml initial concentration, a few of which were toxic to worms and then tested at 0.1 mg/ml or 0.025 mg/ml concentration. Each plate was seeded with 100 μl concentrated E. coli OP50 bacteria

Age-synchronized worms were obtained by treating gravid adults with 2% sodium hypochlorite and 0.5M NaOH to isolate embryos (Lewis Fleming, 1995). These embryos were grown for 30 h at 25° C. At the L3 stage, larvae were incubated with 10 mM (50 mM) 6-OHDA and 2 mM (or 10 mM) ascorbic acid for 1 h with gentle agitation every 10 minutes. (Nass et al., 2002). The worms were then washed and spread onto NGM plates seeded with bacteria (OP50) and scored at time points ranging from 2 to 72 h after 6-OHDA exposure.

Immediately after 6-OHDA treatment, worms containing the transgenes were selected under a fluorescence dissecting microscope, based on the presence of GFP, and transferred to a fresh NGM plate seeded with OP50. For each time point, 30-40 worms were applied to a 2% agarose pad and immobilized with 3 mM levamisole. Worms were examined under a Nikon Eclipse E800 epifluorescence microscope equipped with an Endow GFP filter cube (Chroma Technology, Rockingham, Vt.). For ease of analysis, only the four CEP DA neurons in the head of the worm were scored. A worm was scored as “wild type” when all four CEP neurons were present and their neuronal processes were intact; a worm was scored as having “dendrite blebbing”, “cell body rounding,” or “cell body loss” when at least one of the four neuronal dendrites of cell bodies was defective as described. These experiments were repeated three times, images were captured with a Cool Snap CCD camera (Photometrics, Tucson, Ariz.) driven by MetaMorph software (Universal Imaging, West Chester, Pa.).

Results

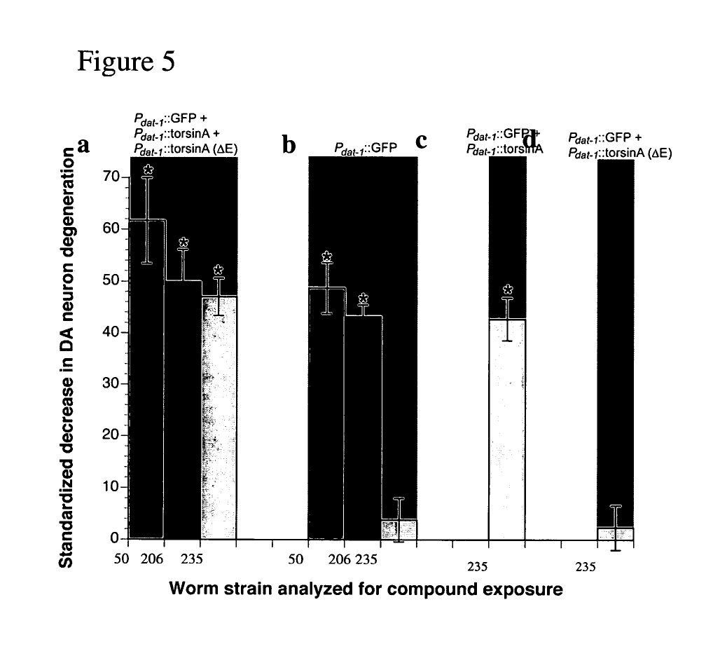

Worms expressing both mutant and wild-type torsins have ˜50% defective dopamine neurons. Primary screening of the library identified 3 molecules that reproducibly reduce dopaminergic neurodegeneration in worms containing P_{dat-1}::GFP+P_{dat-1}::torsinA+Pdat-1::torsinA (ΔE) after 6-OHDA exposure (FIG. 5a).

To elucidate the mechanism by which the small molecule compounds act, all drugs identified in the primary screen were plated with transgenic worms containing the transgene P_{dat-1}::GFP without any torsinA expression. Of the 3 compounds identified in primary screen, 2 drugs, lidocaine HCl (50) and meclofenamic acid sodium salt monohydrate (206) reduced the 6-OHDA-mediated neurodegeneration (FIG. 5b). This result suggests that lidocaine HCl and meclofenamic acid sodium salt monohydrate prevent neurodegeneration through a torsin-independent mechanism.

To explore the mechanism of action for metampicillin sodium salt (235), two transgenic worm strains expressing P_{dat-1}::GFP+P_{dat-1}::torsinA (encoding wt torsinA) or P_{dat-1}::GFP+Pdat-1::torsinA (ΔE) (encoding mutant torsinA) were treated with metampicillin sodium salt prior to 6-OHDA insult. Neuroprotection by metampicillin sodium salt was only afforded in P_{dat-1}::GFP+P_{dat-1}::torsinA worms expressing wt torsinA (FIGS. 5c and 5d). These results indicate that metampicillin sodium salt works through torsin in protecting DA neurons.

Collectively, these data demonstrate that a library of candidate drug compounds may be screened in this C. elegans model for an effect on preventing neurodegeneration associated with reactive oxygen species. Broader classes of these drugs may also be screened using this model to identify other drug compounds that have an effect on preventing neurodegeneration and neuronal loss associated with reactive oxygen species.

### Example 3

### Protection of Neurons in a Model of Neurodegeneration Using a Transgenic C. elegans Overexpressing Tyrosine Hydroxylase

Overexpression of cat-2, the worm homologue for tyrosine hydroxylase, results in increased intraneuronal dopamine production and a characteristic loss of dopaminergic neurons in 75% of transgenic worms as compared to wild type (Cao et al., J Neurosci, 25(1):3801-3812). Co-expression of worm or human torsin proteins reduces the loss of dopaminergic neurons to a slight degree although neuronal degeneration is still present. The purpose of these experiments was to determine the effect of small molecule compounds in the Prestwick library in another different C. elegans model of neurodegeneration.

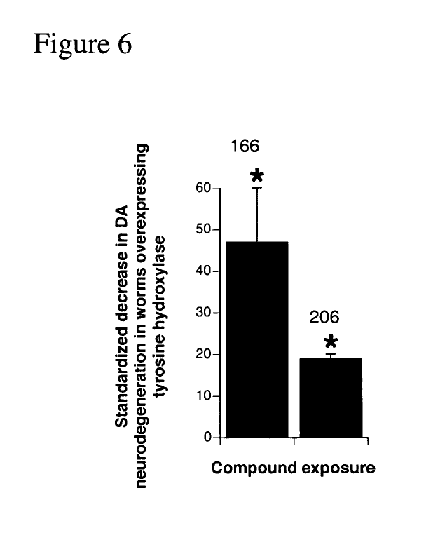

Worms were cultured using the same methods described above. A transgenic worm line expressing P_{dat-1}::CAT-2 expresses a phenotype that results in visible neurodegeneration at all developmental stages in an integrated line, in which only approximately 55% of 7 day old animals maintained all four CEP neurons. Screening experiments using this model of neuroprotection demonstrated that two compounds in particular have neuroprotective actions on dopaminergic neurons overexpressing cat-2. Compounds 166 (mafenide) and 206 (meclofenamic acid sodium salt) both demonstrated a reduction in the standardized decrease in dopaminergic neurodegeneration in the transgenic worms (FIG. 6). These results also demonstrate that compounds having shown little to no neuroprotection in one model for neurodegeneration may still yield a positive result in a different model for neurodegeneration presumably by acting via different mechanisms of action. Compound 166 is an example of one such compound.

### Example 4

### Screening Molecules Related to Compound 235 in a C. elegans Model for Neurodegeneration

Molecules in the Prestwick library that have similar structures and mechanism of action to the compounds identified in the primary screen may be re-analyzed for neuroprotective actions in various models for neurodegeneration. These molecules are listed in Table 3 above.

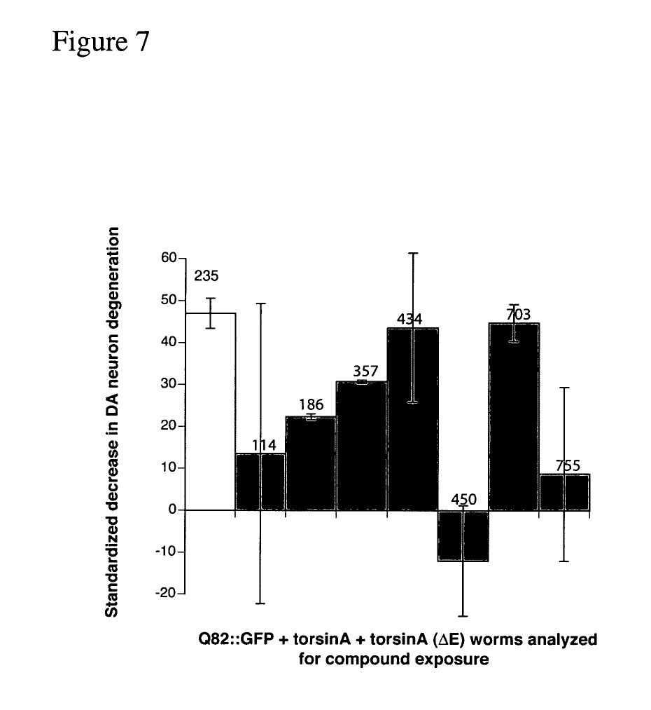

All molecules are coded by Prestwick library number. Of particular importance are compounds related to metampicillin sodium salt (compound 235). Metampicillin is neuroprotective partly by modulating the actions of torsinA protein (FIGS. 5a-5d). While no neuronal loss is observed with dystonia, torsin-dependent mechanisms are involved and therefore compounds that modulate torsin activity are relevant to the treatment of dystonia where expression of a defective mutant torsinA is believed to be responsible for the neuronal dysfunction associated with the disorder. Screening of compounds related to metampicillin in a C. elegans neuroprotection model resulted in variable neuroprotective actions. These data are shown in FIG. 7. While some of the compounds tested did not demonstrate a significant action on neuroprotection in this model, it is possible that other neuroprotection assays may demonstrate actions on preventing neuronal death and degeneration. In particular, cefadroxil and carbenicillin disodium salt (compounds 434 and 703) displayed neuroprotective actions to the same degree as compound 235. Compounds cloxacillin sodium salt and amoxicillin (compounds 186 and 357) also demonstrated neuroprotective actions in this model, albeit to a lesser degree. Experiments showing positive response by the drug candidates were repeated multiple times to confirm the neuroprotective actions. Compounds demonstrating a positive response in this model are easily re-screened with the (Pdat-1::GFP+Pdat-1::torsinA) and (Pdat-1::GFP+Pdat-1::torsinA (ΔE)) models described previously to identify if the actions are also through a torsin-dependent mechanism.

These results demonstrate that a library of candidate drug compounds may be screened in this C. elegans model for neuroprotective actions. Broad classes of drugs, such as the other classes listed in Table 3, may also be screened using this model to identify other drug compounds that have neuroprotective actions.

### Example 5

### Reconfirmation of TorsinA-Dependency in the α-Synuclein Toxicity Assay

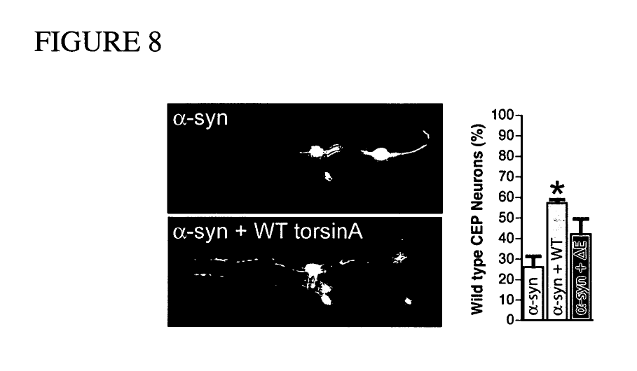

We have previously shown that torsinA can prevent dopamine (“DA”) neuron degeneration resulting from overexpression of α-synuclein in the DA neurons of C. elegans, while torsinA (“ΔE”) has a reduced neuroprotection (Cao et al., J Neurosci, 2005, 25(1): 3801-3812). Specifically, only 26.1±5.3% of the worms expressing P_{dat-1}::GFP+P_{dat-1}::α-synuclein maintained all 4 wildtype CEP DA neurons as 4 day adults while the percentages of the worms expressing P_{dat-1}::GFP+P_{dat-1}::α-synuclein+P_{dat-1}::torsinA and P_{dat-1}::GFP+P_{dat-1}::α-synuclein+P_{dat-1}:: torsinA (ΔE) are 57.3±1.6% and 42.2±7.3%, respectively (Cao et al., J Neurosci, 2005, 25(1): 3801-3812) (FIG. 8).

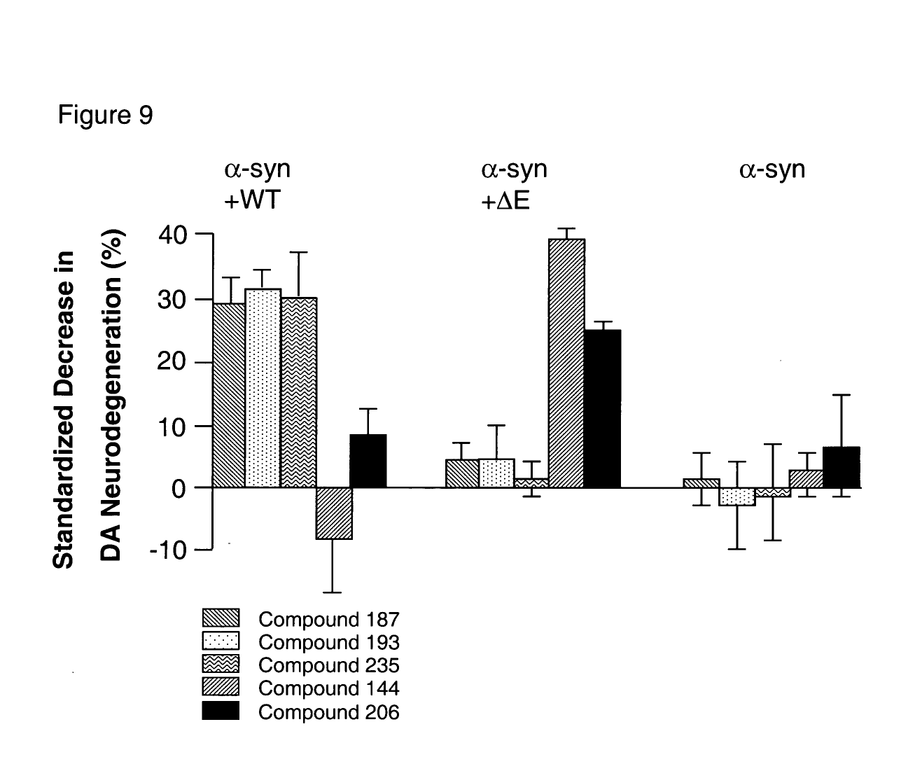

All molecules are coded by Prestwick Library number. TorsinA-dependent compounds identified from the aggregation assay have torsinA-specific effects and, therefore, are likely to function in the same manner in the α-synuclein toxicity assay. All three α-synuclein transgenic lines were exposed to the five torsinA-dependent compounds to determine their torsinA-specificity (FIG. 9). As expected, none of these compounds had any effect in P_{dat-1}::GFP+P_{dat-1}::α-synuclein when torsinA expression is absent. In contrast, metampicillin (compound 235), nalidixic acid (compound 187) and oxolinic acid (compound 193), the three wild type torsinA-dependent compounds, enhanced wild type DA neuron survival in P_{dat-1}::GFP+P_{dat-1}::α-synuclein+P_{dat-1}::torsinA by 30.3±7% (p=0.013), 28.9±4.9% (p=0.005) and 31.4±3.7% (p=0.002), respectively, while loperamide hydrochloride (144) and meclofenamic acid (206), the two torsinA (ΔE)-dependent compounds, failed to show any significant neuroprotection. Conversely, in P_{dat-1}::GFP+P_{dat-1}::α-synuclein+P_{dat-1}:: torsinA (ΔE) worms, metampicillin, nalidixic acid, and oxolinic acid showed no significant neuroprotection, whereas loperamide hydrochloride and meclofenamic acid enhanced DA neuron survival by 39.5±1.4% (p=0.001) and 25±1.2% (p=0.002).

These results demonstrate that an α-synuclein toxicity assay using a C. elegans Parkinson's model (See Cao et al., J Neurosci, 2005, 25(1): 3801-3812) mimics the effect of α-synuclein overexpression as found in the brains of Parkinson's patients. In both humans and nematodes, dopamine neurons die, over time, during the course of aging in response to multiplication of the α-synuclein gene. The C. elegans Parkinson's model clearly demonstrates a therapeutic capacity directly relevant to the human disease state and cross-validates several different model systems for the study of Parkinson's Disease and further establishes that simple model systems can be useful in the investigation of even complex neurodegenerative diseases (See Cooper et al., Science, 2006, 313: 324-328).

### Example 6

### Testing Compounds Functionally Similar to Each TorsinA-Dependent Drug

The activity of functionally similar molecules in addition to those set forth in Table 3 were assayed in a C. elegans model for neuroprotection containing P_{unc-54}::Q82::GFP+P_{unc-54}::torsinA+P_{unc-54}::torsinA (ΔE) to determine if there was significant activity associated with the functionally similar compounds (Table 4).

These results demonstrate that additional functionally similar compounds, as shown in Table 4, demonstrate significant neuroprotective activity. Broad classes of drugs, such as the other classes listed in Table 3, may also be screened using this model to identify other drug compounds that have neuroprotective actions.

TABLE 4
Activity of functionally similar compounds

| | | P-value |
|---|---|---|
| | Functionally similar | (indicating |
| Compound class | compound (Drug name) | significance) |
| Quinolones | Nalidixic acid sodium | p < 0.001 |
| (topoisomerase II | salt (Neggram) | |
| inhibitors) | Oxolinic acid | p < 0.001 |
| | Norfloxacin (Noroxin) | p = 0.003 |
| | Enoxacin (Penetrex) | p < 0.001 |
| Beta-lactams | Ampicillin | p < 0.001 |
| (bacterial | Bacampicillin (Spectrobid) | p = 0.025 |
| transpeptidases) | Metampicillin sodium salt | p < 0.001 |
| | Cyclacillin sodium salt | p = 0.002 |
| | Cloxacillin sodium salt | p = 0.002 |
| | Dicloxyacillin sodium salt | p = 0.055 |
| | Carbenicillin disodium salt | p = 0.003 |
| | Piperacillin sodium salt | p = 0.004 |
| Ca²⁺ | Loperamide hydrochloride | p < 0.001 |
| antagonists | Nifedipine | p < 0.001 |
| | R-(+) BayK86443 | p = 0.027 |
| | Diltiazem hydrochloride | p = 0.01 |
| Anti-inflammatory | Meclofenamic acid sodium | p < 0.001 |
| | salt | |
| | Naproxen | p = 0.019 |
| | Dipyrone | p = 0.003 |

### Example 7

### Lidocaine and Meclofenamic Acid Protect Against 6-OHDA Through Different Mechanisms

To test whether meclofenamic acid or lidocaine can down-regulate DAT-1 protein level independent of torsinA function, we used a transgenic line P_{dat-1}::GFP::DAT-1 previously described (Cao et al., J Neurosci, 2005, 25(1): 3801-3812) where the dat-1 cDNA is fused in-frame with gfp under the control of DA neuron specific promoter to generate a fusion protein between GFP and DAT-1. TorsinA is able to down-regulate GFP::DAT-1 levels, as previously shown by examining the fluorescence intensity and the prevalence of visible GFP expression within transgenic populations (Cao et al., J Neurosci, 2005, 25(1): 3801-3812).

We treated P_{dat-1}::GFP::DAT-1 with meclofenamic acid or lidocaine and found that meclofenamic acid decreased the fluorescence intensity from 1970 A.U. in the control to 1740 A.U. (p=0.029) while the percentage of the worms with both cell body and neuronal process GFP decreased from 70% in the control to 44.7% (p=0.029) (Table 5). Lidocaine decreased the fluorescence intensity in P_{dat-1}::GFP::DAT-1 from 1970 A.U. in the control to 1612 A.U. (p=0.002) while the percentage of the worms with both cell body and neuronal process GFP decreased from 70% in the control to 30% (p=0.0007) (Table 5). These data demonstrate that both lidocaine and meclofenamic acid can down-regulate the level of DAT-1 protein directly.

To ensure that this was not due to a non-specific effect, we tested a compound (ciclopirox ethanolamine) from the Prestwick library that showed no neuroprotection against 6-hydroxydopamine (“6-OHDA”), for its effect on GFP::DAT-1 level. Ciclopirox ethanolamine did not have any significant effect [1923 A.U. of fluorescence intensity (p=0.82) and 66.7% of the worms had both cell body and neuronal process GFP (p=0.71)] compared to controls (Table 5).

To further examine the neuroprotective effect of meclofenamic acid and lidocaine, we used a transgenic line (P_{dat-1}::CAT-2) we previously described (Cao et al., J Neurosci, 2005, 25(1): 3801-3812). Overexpression of cat-2, the C. elegans homolog of the tyrosine hydroxylase (“TH”) gene, causes a high level of DA production and as a result, DA neuron degeneration is observed (Cao et al., J Neurosci, 2005, 25(1): 3801-3812). We tested lidocaine in P_{dat-1}::CAT-2; it did not have any significant neuroprotective effect against CAT-2-induced neurodegeneration (−7.9%±5%, p=0.6), suggesting that its observed neuroprotective effect against 6-OHDA is solely the result of the down-regulation of DAT-1 levels. We also tested whether meclofenamic acid was able to suppress CAT-2-induced neurodegeneration. Notably, it had a neuroprotective effect of 19±1.1% (p=0.002). Therefore, meclofenamic acid can protect DA neurons from degeneration produced by overexpression of the dopamine precursor, tyrosine hydroxylase.

These results demonstrate that lidocaine and meclofenamic acid exert neuroprotective effects against the neurotoxin 6-OHDA through different mechanisms.

TABLE 5
Effect of compounds on P_{dat-1}::GFP::DAT-1 expression within DA neurons

| | None | Lidocaine | Meclofenamic acid | Ciclopirox |
|---|---|---|---|---|
| Chemical | (water control) | hydrochloride^{a} | sodium salt^{a} | etnanolamine^{a} |
| Mean pixel intensity of cell | 1970 ± 80 | 1612 ± 78 | 1740 ± 64 | 1923 ± 85 |
| bodies ± SEM (A.U.) | (n = 69)^{b} | (n = 57)^{b,c} | (n = 48)^{b,c} | (n = 64)^{b} |
| Neurons with cell body and | 70% | 30%^{d} | 44.7%^{d} | 66.7% |
| dendritic/axonal GFP | | | | |
| Neurons with cell body | 30% | 70%^{e} | 51.1%^{e} | 32.3% |
| GFP only | | | | |
| Worms with no GFP | 0% | 0% | 4.2% | 0% |

n represents the number of cell bodies analyzed from 40-47 worms/compound exposure (see footnote b)
^{a}Lidocaine hydrochloride, meclofenamic acid, and ciclopirox ethanolamine were dissolved in water at 1.85 mM, 1.49 mM, and 1.86 mM, respectively.
^{b}Forty to forty-seven worms were analyzed from each strain in which one to two cell bodies from each worm were analyzed for pixel intensity.
^{c}mean pixel intensity of cell bodies from P_{dat-1}::GFP::DAT-1 worms exposed to solvent only or exposed to ciclopirox ethanolamine is significantly different from worms exposed to either lidocaine hydrochloride (p = 0.002) or meclofenamic acid (p = 0.029).
^{d}average percentage of neurons with cell body and dendritic/axonal GFP is significantly reduced in worms exposed to lidocaine hydrochloride (p = 0.0007) or meclofenamic acid (p = 0.029) when compared to worms exposed only to solvent or ciclopirox ethanolamine
^{e}average percentage of neurons with cell body GFP only is significantly enhanced in worms exposed to lidocaine hydrochloride or meclofenamic acid when compared to worms exposed only to solvent or ciclopirox ethanolamine

All documents referred to in this specification are herein incorporated by reference.

Various modifications and variations to the described embodiments of the inventions will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

We claim:

1. A method for preventing neurodegeneration and neuronal loss associated with protein misfolding or aggregation by administering a composition comprising an effective amount of a small molecule compound to a mammal in need of treatment for preventing neurodegeneration and neuronal loss associated with protein misfolding or aggregation, wherein the small molecule compound has the effect of preventing neurodegeneration and neuronal loss associated with protein misfolding or aggregation, and
wherein the small molecule compound comprises a topoisomerase II inhibitor, bacterial transpeptidase inhibitor, calcium channel antagonist, cyclooxygenase inhibitor, folic acid synthesis inhibitor, or sodium channel blocker and functional analogues thereof.

2. The method of claim 1 wherein the topoisomerase II inhibitor comprises lomefloxacin, cinoxacin, amsacrine, etoposide, teniposide, oxolinic acid, nalidixic acid, suramin, merbarone, genistein, epirubicin HCl, ellipticine, doxorubicin, aurintricarboxylic acid or pharmaceutically acceptable salts thereof.

3. The method of claim 2 wherein the topoisomerase II inhibitor comprises oxolinic acid or nalidixic acid.

4. The method of claim 3 wherein the oxolinic acid is administered at a dosage between about 10 mg/kg and about 40 mg/kg.

5. The method of claim 3 wherein the nalidixic acid is administered at a dosage between about 1 gram/day and about 5 grams/day.

6. The method of claim 1 wherein the bacterial transpeptidase inhibitor comprises ampicillin, cloxacillin, piperacillin, amoxicillin, cefadroxil, dicloxyacillin, carbenicillin, penicillin, metampicillin, amoxicillin, cefoxatin or pharmaceutically acceptable salts thereof.

7. The method of claim 6 wherein the bacterial transpeptidase inhibitor comprises metampicillin.

8. The method of claim 7 wherein the metampicillin is administered at a dosage between about 250 mg/kg and about 500 mg/kg every 8 hours.

9. The method of claim 1 wherein the calcium channel blocker comprises nimodipine, diproteverine, verapamil, nitrendipine, diltiazem, mioflazine, loperamide, flunarizine, bepridil, lidoflazine, CERM-196, R-58735, R-56865, ranolazine, nisoldipine, nicardipine, PN200-110, felodipine, amlodipine, R-(−)-202-791, or R-(+) Bay K-8644 or pharmaceutically acceptable salts thereof.

10. The method of claim 9 wherein the calcium channel blocker comprises loperamide.

11. The method of claim 10 wherein the loperamide is administered at a dosage between about 1 mg/kg and about 5 mg/kg.

12. The method of claim 1 wherein the cyclooxygenase inhibitor comprises naproxen, flufenamic acid, tolfenamic acid, fenbufen, ketoprofen, phenacetin, dipyrone, flurbiprofen, meclofenamide, piroxicam, indomethacine or pharmaceutically acceptable salts thereof.

13. The method of claim 12 wherein the cyclooxygenase inhibitor comprises meclofenamide.

14. The method of claim 13 wherein the meclofenamide is administered at a dosage between about 25 mg/kg and about 75 mg/kg.

15. The method of claim 1 wherein the folic acid synthesis inhibitor comprises sulfonamides, dapsone, trimethoprim, diaveridine, pyrimethamine, methotrexate, or pharmaceutically acceptable salts thereof.

16. The method of claim 15 wherein the folic acid synthesis inhibitor comprises mafenide.

17. The method of claim 16 wherein the mafenide is administered at a dosage between about 250 mg/kg and about 750 mg/kg every 6 hours.

18. The method of claim 1 wherein the sodium channel blocker comprises lidocaine, dyclonine HCl, mexilitine, phenyloin, ketamine, flecainide, amantadine or pharmaceutically acceptable salts thereof.

19. The method of claim 18 wherein the sodium channel blocker comprises dyclonine HCl.

20. The method of claim 19 wherein the dyclonine HCl is administered at a dosage between about 2 mg/kg and about 3 mg/kg every 2 hours.

21. The method of claim 1 wherein the small molecule compound is administered by inhalation, transdermal, oral, rectal, transmucosal, intestinal, or parenteral routes.

22. The method of claim 1, wherein the small molecule compound is administered to the mammal after the onset of neurodegeneration and neuronal loss associated with protein misfolding or aggregation.

23. The method of claim 1 wherein the small molecule compound modulates the activity of a torsin protein.

24. The method of claim 23 wherein the small molecule compound modulates the activity of a wild-type torsinA protein.

25. The method of claim 23 wherein the small molecule compound modulates the activity of a mutant torsinA protein.

26. The method of claim 1 wherein the protein misfolding or aggregation is associated with a neurodegenerative disease comprising amyotrophic lateral sclerosis, Alzheimer's disease, Parkinson's disease, prion disease, polyglutamine expansion diseases, spinocerebellar ataxia, spinal and bulbar muscular atrophy, spongiform encephalopathy, tauopathy, Huntington's disease, or dystonia.

27. A method for preventing neurodegeneration and neuronal loss associated with reactive oxygen species by administering a composition comprising an effective amount of a small molecule compound to a mammal in need of treatment for preventing neurodegeneration and neuronal loss associated with reactive oxygen species, wherein the small molecule compound has the effect of preventing neurodegeneration and neuronal loss associated with reactive oxygen species, and
wherein the small molecule compound comprises topoisomerase II inhibitors, bacterial transpeptidase inhibitors, calcium channel antagonists, cyclooxygenase inhibitors, folic acid synthesis inhibitors, or sodium channel blockers and functional analogues thereof.

28. The method of claim 27 wherein the folic acid synthesis inhibitor comprises mafenide HCl, sulfacetamide sodic hydrate, sulfadiazine, sulfaguanidine, sulfathiazole, sulfamethoxazole, sulfabenzamide or functional analogues thereof.

29. The method of claim 28 wherein the folic acid synthesis inhibitor comprises mafenide.

30. The method of claim 29 wherein the mafenide is administered at a dosage between about 250 mg/kg and about 500 mg/kg.

31. The method of claim 27 wherein the sodium channel blocker comprises lidocaine, dyclonine HCl, mexilitine, phenyloin, ketamine, flecainide, amantadine or pharmaceutically acceptable salts thereof.

32. The method of claim 31 wherein the sodium channel blocker comprises lidocaine.

33. The method of claim 32 wherein the lidocaine is administered at a dosage between about 1 mg/kg and about 50 mg/kg.

34. The method of claim 27 wherein the cyclooxygenase inhibitor comprises flurbiprofen, meclofenamide, piroxicam, indomethacine or pharmaceutically acceptable salts thereof.

35. The method of claim 34 wherein the cyclooxygenase inhibitor comprises meclofenamide.

36. The method of claim 35 wherein the meclofenamide is administered at a dosage between about 25 mg/kg and about 75 mg/kg.

37. The method of claim 27 wherein the bacterial transpeptidase inhibitor comprises ampicillin, penicillin, cefadroxil, amoxicillin, piperacillin, cloxacillin carbenicillin, metampicillin, dicloxyacillin, amoxicillin, cefoxatin or pharmaceutically acceptable salts thereof.

38. The method of claim 37 wherein the bacterial transpeptidase inhibitor comprises metampicillin.

39. The method of claim 38 wherein the metampicillin is administered at a dosage between about 250 mg/kg and about 500 mg/kg every 8 hours.

40. The method of claim 27 wherein the small molecule compound is administered by inhalation, transdermal, oral, rectal, transmucosal, intestinal, or parenteral routes.

41. The method of claim 27 wherein the reactive oxygen species is associated with a neurodegenerative disease comprising amyotrophic lateral sclerosis, Alzheimer's disease, Parkinson's disease, prion diseases, polyglutamine expansion diseases, spinocerebellar ataxia, spinal and bulbar muscular atrophy, spongiform encephalopathy, tauopathy, Huntington's disease, or dystonia.

42. The method of claim 27 wherein the small molecule compound further comprises a reactive oxygen species scavenger or at least one neurotrophic factor.

43. The method of claim 42, wherein the reactive oxygen species scavenger comprises coenzyme Q, vitamin E, vitamin C, pyruvate, melatonin, niacinamide, N-acetylcysteine, glutathione, or a nitrone.

44. The method of claim 27, wherein the small molecule compound is administered to the mammal after the onset of neurodegeneration and neuronal loss associated with reactive oxygen species.

45. The method of claim 27, wherein the small molecule compound modulates the neuroprotective activity of a torsin protein.

46. The method of claim 45, wherein the small molecule compound indirectly modulates the neuroprotective activity of the torsin protein.

47. The method of claim 27 wherein the neurons express tyrosine hydroxylase.

48. The method of claim 47 wherein the neurons are dopaminergic.
